(19) United States
(12) Patent Application Publication
(10) Pub. No.: US 2025/0269164 A1
(43) Pub. Date: Aug. 28, 2025

(54) Removable Dust Cap And Tamper-Evident Feature For Syringe

(71) Applicant: Bayer Healthcare LLC, Whippany, NJ (US)

(72) Inventors: Mikayla Ferchaw, Pittsburgh, PA (US); Sean Kelly McKillop, Pittsburgh, PA (US); David Berry, Kittanning, PA (US); Russell Tartock, Salisbury, NC (US); Corey Savannah, Butler, PA (US); Gary Gogolin, Tampa, FL (US); James Brown, Wesley Chapel, FL (US); Steven Stricko, Trafford, PA (US); Kevin Cowan, Allison Park, PA (US)

(21) Appl. No.: 18/859,528

(22) PCT Filed: Apr. 27, 2023
(86) PCT No.: PCT/US2023/020171

§ 371 Date: Oct. 23, 2024

Related U.S. Application Data
(60) Provisional application No. 63/336,284, filed on Apr. 28, 2022.
(60) Provisional application No. 63/415,488, filed on Oct. 12, 2022.

(51) Int. Cl. A61M 39/10; A61M 5/14; A61M 5/145; A61M 5/50

(52) U.S. Cl. CPC A61M 39/1011; A61M 5/1413; A61M 5/1452; A61M 5/5086

(57) ABSTRACT

A fluid path connector assembly for a medical fluid delivery system includes a connector element having a body, a lumen extending through the body, and a pair of flexible legs connected to the body, each of the flexible legs having a flange. The connector assembly further includes a dust cap removably connected to the connector element. The dust cap includes a dust cap body positioned between the flexible legs to enclose the lumen, and an engagement element at a distal end of the dust cap body. The engagement element is in contact with the flanges of the flexible legs to prevent disengagement of the dust cap from the connector element while allowing ready removal of the dust cap by medical personnel. A removable/disconnectable tamper resistant and/or tamper-evident and/or tamper resistant feature connecting the dust cap to the connector element is also disclosed.

### CROSS-REFERENCE TO RELATED APPLICATION

This application claims priority to U.S. Provisional Application No. 63/336,284, filed on Apr. 28, 2022, and U.S. Provisional Application No. 63/415,488, filed on Oct. 12, 2022, the disclosures of which are incorporated herein by reference in their entirety.

### BACKGROUND OF THE DISCLOSURE

### Field of the Disclosure

The present disclosure relates to dust caps, connectors, syringes, and syringe and connector systems for use in fluid delivery systems, and, especially, to dust caps and tamper-evident features for connector systems on syringes for use in medical fluid delivery systems in which one or more fluids are delivered to a patient.

### Description of Related Art

In many medical procedures, such as drug delivery, it is desirable to inject a liquid into a patient. Numerous types of liquids, such as contrast media (often referred to simply as “contrast”) and/or saline, may be injected into a patient during diagnostic and therapeutic procedures. In some medical procedures, for example, angiography, computed tomography (CT), ultrasound, magnetic resonance imaging (MRI), nuclear medicine, and positron emission tomography (PET), it is necessary to deliver a liquid, such as contrast, in a timed fashion under high pressure, such as up to 1200 psi. Injectors suitable for these applications may use a relatively large volume syringe and are capable of producing relatively large flow rates.

For medical syringes and associated disposable features, it is important that the various components of the fluid path to the patient, such as syringes, connectors, fluid lines, and other components for delivering fluid to the patient, remain sterile prior to connection to the patient. Inadvertent contamination of the fluid path during manufacture, shipping, unpackaging, and/or setup may pose a risk to the patient. Ensuring that the fluid path, such as the interior volume of a syringe, remains sterile requires new designs and configurations for caps of the syringe, such as dust caps. Further, it is important that the user be sure that the various components are sterile and have not been contaminated, such as by prior use or tampering (e.g., opening of the packaging and/or contamination of a sterile surface of the component).

Medical personnel must connect and/or disconnect fluid delivery systems in a relatively short time and under stressed and/or emergency conditions, so it is important that the various components of the fluid path are easy and intuitive to use. It is thus desirable to develop syringe dust caps that are configured for ready use but ensure that the fluid pathway remains sterile prior to connection to the patient.

It is important for the various components of the fluid path to be unused or otherwise tampered with prior to their initial use to ensure sterility of the fluid path to the patient. Accordingly, it is desirable to develop syringe dust caps that include one or more tamper evident features such that the sterility of the fluid path components can be maintained.

### SUMMARY OF THE DISCLOSURE

In one example of the present disclosure, embodiments of dust caps are described. The various embodiments of the dust cap are configured to ensure that regions of the syringe that contact the fluid to be injected into the patient remain sterile during shipping, unpackaging, and setup to avoid inadvertent contamination of the fluid pathway and potentially put the patient at risk. The dust caps may engage features associated with the syringe, such as features of a connector system at the nozzle of the syringe to ensure that the dust cap protects and retains the sterility of the inner volume of the syringe and associated connector features that may contact a medical fluid. In addition, the dust cap configurations may be configured to be readily removed from the connector by the medical personnel prior to connection of fluid path components for a medical injection procedure. In certain embodiments, the dust cap may also include one or more tamper evident features that allow the medical personnel to determine if the dust cap seal has been tampered with, the syringe previously used, or otherwise damaged, for example during shipping, such that the sterility of the fluid path components may be compromised. Various embodiments and features of dust caps for use with a syringe, for example a high pressure angiography syringe, CT syringe, or MRI syringe for use with a fluid injector system are described.

In some embodiments, provided is a fluid path connector assembly for a medical fluid delivery system. The fluid path connector assembly may include a connector element having a body with a perimeter skirt surrounding the connector element, and a lumen extending through the body. The connector element further may include a first flexible leg connected to the body, the first flexible leg having a first flange, and a second flexible leg connected to the body, the second flexible leg having a second flange. The fluid path connector assembly further may include a dust cap removably connected to the connector element. The dust cap may include a dust cap body positioned between the first flexible leg and the second flexible leg and surrounding the body of the connector element to enclose the lumen. The dust cap body may have a proximal end and distal end, and an engagement element at the distal end of the dust cap body. The engagement element may include a sloping member defined by a distal edge of the dust cap body. The engagement element may be in contact with the first flange of the first flexible leg and the second flange of the second flexible leg of the connector element to prevent disengagement of the dust cap from the connector element. The first flange of the first flexible leg and the second flange of the second flexible leg of the connector element may be configured to contact the sloping member when the dust cap is moved distally relative to the connector element such that the first flexible leg and the second flexible leg expand radially outward to permit removal of the dust cap from the connector element.

In some embodiments, the sloping member may extend around at least a portion of an outer perimeter of the distal edge of the dust cap body. The sloping member may be a rounded circumferential edge or a chamfered circumferential edge. The engagement element may include a ledge configured to contact the first flange of the first flexible leg and the second flange of the second flexible leg of the connector element.

In some embodiments, a grasping tab may be connected to the distal end of the dust cap body. The grasping tab may include one or more gripping elements configured to facilitate removal of the dust cap from the connector element.

In some embodiments, the dust cap body may be rotatable relative to the body of the connector element about a longitudinal axis of the dust cap. When the dust cap is connected to the connector element, air pressure within the lumen of the connector element may equalize with the outside atmospheric pressure while maintaining a sterility of the connector element. In some embodiments, a tortuous fluid path is defined between the dust cap body and the circumferential skirt of the body of the connector element to permit equalization of pressure within the lumen of the connector element while preventing a passage of contaminants into the lumen of the connector element. The connector element may be configured to fluidly connect to at least one component selected from the group consisting of: a syringe, a spike, a fluid tube set, and a bulk fluid container.

In some embodiments, the fluid path connector assembly may include a removeable/disconnectable tamper resistant and/or tamper evident feature connecting the dust cap to the connector element. When present, the tamper resistant and/or tamper evident feature may be configured to prevent removal of the dust cap from the connector element, indicate prior tampering with the connection between the connector element and the dust cap, and/or prevent or indicate reinstallation of the dust cap onto the connector element after the dust cap is removed from the connector element.

In some embodiments, a fluid path connector assembly may include a connector element having a body with a circumferential skirt surrounding the connector element, and a lumen extending through the body. The connector element further may include a first flexible leg connected to the body, and a second flexible leg connected to the body. The fluid path connector assembly further may include a dust cap removably connected to the connector element. The dust cap may include a dust cap body positioned between the first flexible leg and the second flexible leg and surrounding the body of the connector element to enclose the lumen. The fluid path connector assembly further may include a removeable/disconnectable tamper resistant and/or tamper evident feature connecting the dust cap to the connector element. When present, the tamper resistant and/or tamper evident feature may be configured to prevent removal of the dust cap from the connector element, indicate prior tampering with the connection between the connector element and the dust cap, and/or prevent or indicate reinstallation of the dust cap onto the connector element after the dust cap is removed from the connector element.

In some embodiments, the tamper resistant and/or tamper evident feature may include a polymeric shrink wrap or a paper wrap at least partially enclosing the connector element and the dust cap. The polymeric shrink wrap or the paper wrap may have at least one removal feature for removing the shrink wrap from the fluid path connector assembly. The removal feature may include a tearable pre-cut, a gripable tab, or perforation feature for removing the tamper resistant and/or tamper evident feature.

In some embodiments, the tamper resistant and/or tamper evident feature may include a cap at least partially enclosing the connector element and the dust cap. The cap may be secured to a syringe via a tearable strip.

In some embodiments, the tamper resistant and/or tamper evident feature may include a first portion connected to the connector element and a second portion connected to the first portion and the dust cap. The first portion may include a pull tab configured for removing the first portion from the connector element. The second portion may be configured to break from the first portion or the dust cap with rotation of the dust cap about a longitudinal axis.

In some embodiments, the tamper resistant and/or tamper evident feature may be a removable band surrounding the first flexible leg and the second flexible leg. The band restricts movement of the first flexible leg and the second flexible leg to prevent removal of the dust cap when the removable band is attached to the fluid path connector assembly.

In some embodiments, the tamper resistant and/or tamper evident feature may have a first end connected to the connector element and a second end connected to the dust cap. The first end may be configured to break or separate from the connector element with rotation of the dust cap about a longitudinal axis.

In some embodiments, the tamper resistant and/or tamper evident feature may include a tearable pull tab monolithically formed with the dust cap. The dust cap may include a first flexible arm connected to the dust cap body and configured to removably engage the first flexible leg of the connector element, and a second flexible arm connected to the dust cap body and configured to removably engage the second flexible leg of the connector element. The first flexible arm and the second flexible arm of the dust cap may prevent reinstallation of the dust cap on the connector element after the dust cap is removed from the connector element.

In some embodiments, the dust cap body may include a first portion and a second portion connected together and separable from each other along a separation line substantially parallel with a longitudinal axis of the dust cap. The first portion and the second portion may have at least one reinsertion prevention element to prevent reinstallation of the dust cap on the connector element after the dust cap is removed from the connector element.

In some embodiments, the dust cap body may include a first groove configured to receive at least a portion of the first flexible leg and a second groove configured to receive at least a portion of the second flexible leg.

In some embodiments, when the dust cap and the tamper resistant and/or tamper evident feature are connected to the connector element, air pressure within the lumen of the connector element may equalize with the outside atmospheric pressure while maintaining a sterility of the connector element. A tortuous fluid path may be defined between the tamper resistant and/or tamper evident feature, the dust cap body, and the body of the connector element to permit equalization of pressure within the lumen of the connector element while preventing a passage of contaminants into the lumen of the connector element. The connector element may be configured to fluidly connect to component selected from the group consisting of: a syringe, a spike, a fluid tube set, and a bulk fluid container.

In some embodiments, the fluid path connector assembly further may include an engagement element at the distal end of the dust cap body. The engagement element may include a sloping member defined by a distal edge of the dust cap body. The engagement element may be in contact with the first flange of the first flexible leg and the second flange of the second flexible leg of the connector element to prevent disengagement of the dust cap from the connector element. The first flange of the first flexible leg and the second flange of the second flexible leg of the connector element may be configured to contact the sloping member when the dust cap is moved distally relative to the connector element such that the first flexible leg and the second flexible leg expand radially outward to permit removal of the dust cap from the connector element.

In some embodiments, the sloping member may extend around at least a portion of an outer perimeter of the distal edge of the dust cap body. The sloping member may be a rounded circumferential edge, or a chamfered circumferential edge. The engagement element may include a ledge configured to contact the first flange of the first flexible leg and the second flange of the second flexible leg of the connector element.

### BRIEF DESCRIPTION OF THE DRAWINGS

FIG. 1 is a perspective view of a fluid path connector assembly associated with a syringe according to one example of the present disclosure shown in a disconnected position;

FIG. 2 is a side view of the fluid path connector assembly of FIG. 1 in the disconnected position;

FIG. 3 is a perspective view of the fluid path connector assembly of FIG. 1 in a connected position;

FIG. 4 is a side view of the fluid path connector assembly of FIG. 1 in the connected position;

FIG. 5 is a cross-sectional view of a connector element of the fluid path connector assembly of FIG. 1;

FIG. 6 is an exploded cross-sectional view of the fluid path connector assembly with a dust cap shown in a disconnected position;

FIG. 7 is a perspective view of a fluid path connector assembly with a dust cap associated with a syringe according to one example of the present disclosure shown in a connected position;

FIG. 8 is a cross-sectional view of the fluid path connector assembly of FIG. 6 with a dust cap shown in a connected position;

FIG. 9 is a perspective view of a fluid path connector assembly and a tamper resistant and/or tamper evident feature according to one example of the present disclosure;

FIG. 10 is a perspective view of a fluid path connector assembly and a tamper resistant and/or tamper evident feature according to one example of the present disclosure;

FIG. 11 is a perspective view of a fluid path connector assembly and a tamper resistant and/or tamper evident feature according to one example of the present disclosure;

FIG. 12A is a perspective view of a fluid path connector assembly and a tamper resistant and/or tamper evident feature according to one example of the present disclosure;

FIG. 12B is a perspective view of a dust cap and a tamper resistant or tamper evident seal of the fluid path connector assembly of FIG. 12A;

FIG. 13A is a perspective view of a fluid path connector assembly and a tamper resistant and/or tamper evident feature according to one example of the present disclosure;

FIG. 13B is a detailed perspective view of an attachment point between a dust cap and a connector element of the fluid path connector assembly of FIG. 13A;

FIG. 13C is a perspective view of the fluid path connector assembly of FIG. 13A installed on a syringe;

FIG. 14A is a perspective view of a dust cap of a fluid path connector assembly having a tamper resistant and/or tamper evident feature according to one example of the present disclosure shown in a disassembled state;

FIG. 14B is a perspective view of the dust cap of FIG. 14A in an assembled state;

FIGS. 14C-14E are side views of the dust cap of FIG. 14A installed on a syringe;

FIG. 15A is a perspective view of a dust cap of a fluid path connector assembly having a tamper resistant and/or tamper evident feature according to one example of the present disclosure shown in a disassembled state;

FIG. 15B is a perspective view of the dust cap of FIG. 15A during installation on a syringe;

FIG. 16A is a perspective view of a dust cap of a fluid path connector assembly having a tamper resistant and/or tamper evident feature according to one example of the present disclosure shown in a disassembled state;

FIG. 16B is a perspective view of the dust cap of FIG. 16A during assembly on a syringe;

FIGS. 16C-16E show the dust cap of FIG. 16A installed on the syringe;

FIG. 17A is a perspective view of a dust cap of a fluid path connector assembly having a tamper resistant and/or tamper evident feature in an example of the present disclosure;

FIG. 17B is a perspective view of the dust cap of FIG. 17A installed on a syringe;

FIG. 17C is a side cross-sectional view of the dust cap of FIG. 17A on the syringe;

FIG. 18A is a perspective view of a dust cap of a fluid path connector assembly having a tamper resistant and/or tamper evident feature in an example of the present disclosure;

FIG. 18B is a perspective view of the dust cap of FIG. 18A installed on a syringe;

FIG. 19A is a bottom perspective view of a dust cap of a fluid path connector assembly having a tamper resistant and/or tamper evident feature according to one example of the present disclosure;

FIG. 19B is a top perspective view of the dust cap shown in FIG. 19A;

FIG. 19C is a bottom perspective view of a dust cap of a fluid path connector assembly having a tamper resistant and/or tamper evident feature according to one example of the present disclosure;

FIG. 19D is a top perspective view of the dust cap shown in FIG. 19C;

FIGS. 20A-20D are side cross-sectional (FIGS. 20A and 20C) and perspective views (FIGS. 20B and 20D) of the embodiments of the dust caps of FIGS. 19A-19B installed on a fluid path connector assembly and syringe;

FIG. 21A is a top perspective view of a dust cap for a fluid path connector assembly having a tamper resistant and/or tamper evident feature in an example of the present disclosure;

FIG. 21B is a bottom perspective view of the dust cap shown in FIG. 21A;

FIG. 21C is a perspective view of the dust cap shown in FIG. 21A with a connector element;

FIGS. 21D-21E show the dust cap and connector element of FIG. 21C installed on a syringe in a side perspective view (FIG. 21D) and a top view (FIG. 21E);

FIGS. 22A-22B are top views of a tamper resistant and/or tamper evident feature according to one example of the present disclosure;

FIG. 23 is a perspective view of a dust cap of a fluid path connector assembly having a tamper resistant and/or tamper evident feature for one example of the present disclosure;

FIG. 24 is a perspective view of a dust cap of a fluid path connector assembly having a tamper resistant and/or tamper evident feature for one example of the present disclosure;

for one is a perspective view of a dust cap of a fluid path connector assembly having a tamper resistant and/or tamper evident feature for one example of the present disclosure;

FIG. 26 is a perspective view of a dust cap of a fluid path connector assembly having a tamper resistant and/or tamper evident feature for one example of the present disclosure;

FIG. 27 is a perspective view of a dust cap of a fluid path connector assembly having a tamper resistant and/or tamper evident feature for one example of the present disclosure;

FIG. 28 is a perspective view of a dust cap of a fluid path connector assembly having a tamper resistant and/or tamper evident feature for one example of the present disclosure;

FIG. 29A is a top view of a dust cap of a fluid path connector assembly having a tamper resistant and/or tamper evident feature according to one example of the present disclosure shown in a first or unlocked position;

FIG. 29B is a top view of the dust cap of FIG. 29A shown in a second or locked position;

FIG. 30A is a side view of a dust cap of a fluid path connector assembly having a tamper resistant and/or tamper evident feature according to one example of the present disclosure shown in a first or unlocked engaged position;

FIG. 30B is a side view of the dust cap of FIG. 30A shown in a second or locked removed position;

FIG. 31 is a perspective view of a dust cap of a fluid path connector assembly having a tamper resistant and/or tamper evident feature for one example of the present disclosure;

FIG. 32A is a top view of a dust cap of a fluid path connector assembly having a tamper resistant and/or tamper evident feature according to one example of the present disclosure shown in a first or unlocked position; and

FIG. 32B is a top view of the dust cap of FIG. 32A shown in a second or locked position.

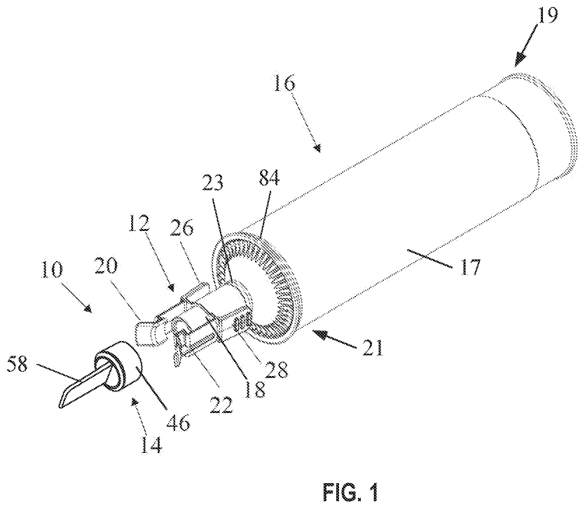

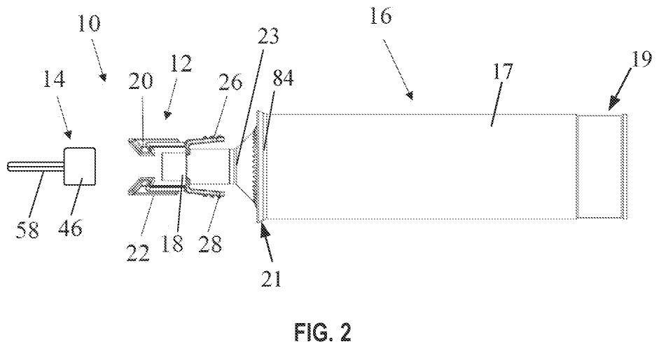

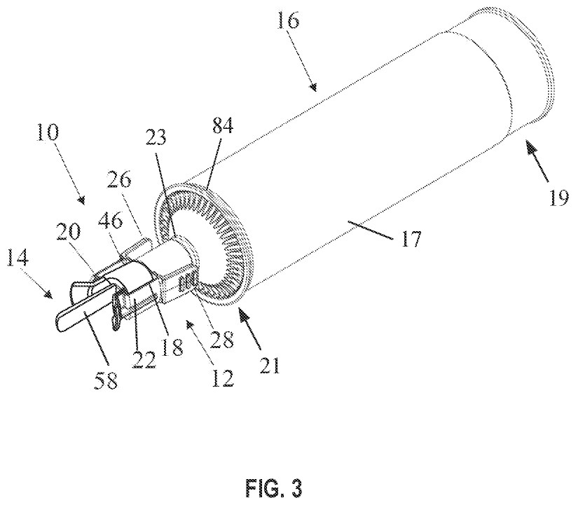

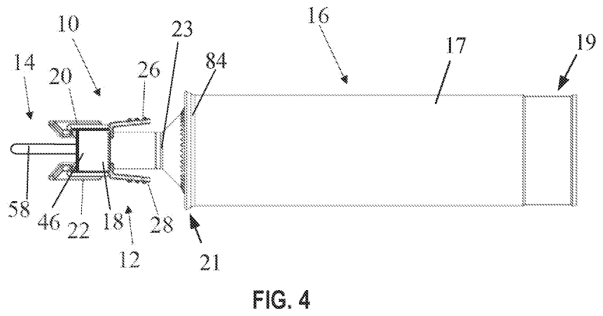

In FIGS. 1-32B, like characters refer to the same components and elements, as the case may be, unless otherwise stated.

The illustrations generally show preferred and non-limiting aspects of the systems and methods of the present disclosure. While the description presents various aspects of the devices, it should not be interpreted in any way as limiting the disclosure. Furthermore, modifications, concepts, and applications of the disclosure's aspects are to be interpreted by those skilled in the art as being encompassed, but not limited to, the illustrations and description provided herein.

### DESCRIPTION OF THE DISCLOSURE

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

As used herein, the singular form of “a”, “an”, and “the” includes plural referents unless noted otherwise. Spatial or directional terms, such as “left”, “right”, “inner”, “outer”, “above”, “below”, and the like, relate to the embodiments or aspects as shown in the drawing figures and are not to be considered as limiting as the embodiments or aspects can assume various alternative orientations. All numbers used in the specification and claims are to be understood as being modified in all instances by the term “about.” By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements. All documents referred to herein are “incorporated by reference” in their entirety. The term “at least” is synonymous with “greater than or equal to.” The term “not greater than” is synonymous with “less than or equal to.” The term “includes” is synonymous with “comprises.”

Some non-limiting embodiments or aspects may be described herein in connection with thresholds. As used herein, satisfying a threshold may refer to a value being greater than the threshold, greater than or equal to the threshold, lower than the threshold, less than or equal to the threshold, equal to the threshold, etc.

As used herein, “at least one of” is synonymous with “one or more of”. For example, the phrase “at least one of A, B, or C” means any one of A, B, or C, or any combination of any two or more of A, B, or C. For example, “at least one of A, B, or C” includes A alone; or B alone; or C alone; or A and B; or A and C; or B and C; or all of A, B, and C.

When used in relation to a component of a fluid delivery system, such as a fluid reservoir, a syringe, a connector, a dust cap, or a fluid line, the term “distal” refers to a portion of said component nearest to a patient. When used in relation to a component of an injector system, such as a fluid reservoir, a syringe, a connector, a dust cap, or a fluid line, the term “proximal” refers to a portion of said component nearest to the injector of the injector system (i.e., the portion of said component farthest from the patient). When used in relation to a component of a fluid delivery system such as a fluid reservoir, a syringe, a connector, a dust cap, or a fluid line, the term “upstream” refers to a direction away from the patient and towards the injector in relation to the normal flow of fluid of the injector system. When used in relation to a component of a fluid delivery system such as a fluid reservoir, a syringe, a connector, a dust cap, or a fluid line, the term “downstream” refers to a direction towards the patient and away from the injector in relation to the normal flow of fluid of the fluid delivery system.

The term “radial” and related terms refer generally to a direction normal to a longitudinal axis of a syringe, a connector, a dust cap, or other component of an injector system However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting.

FIGS. 1-4 illustrate a fluid path connector assembly 10 associated with a syringe 16 according to one example of the present disclosure. The fluid path connector assembly 10 may include a connector element 12 and a dust cap 14 that are configured to connect to one another. In one example of the present disclosure, the connector element 12 may be operatively connected to the syringe 16. In further examples of the present disclosure, the connector element 12 may be configured to fluidly connect to a spike, a fluid tube set, or a bulk fluid container. Various examples of the fluid path connector assembly 10 are described in PCT International Application Publication No. WO2021/168076, the disclosure of which is incorporated by reference herein.

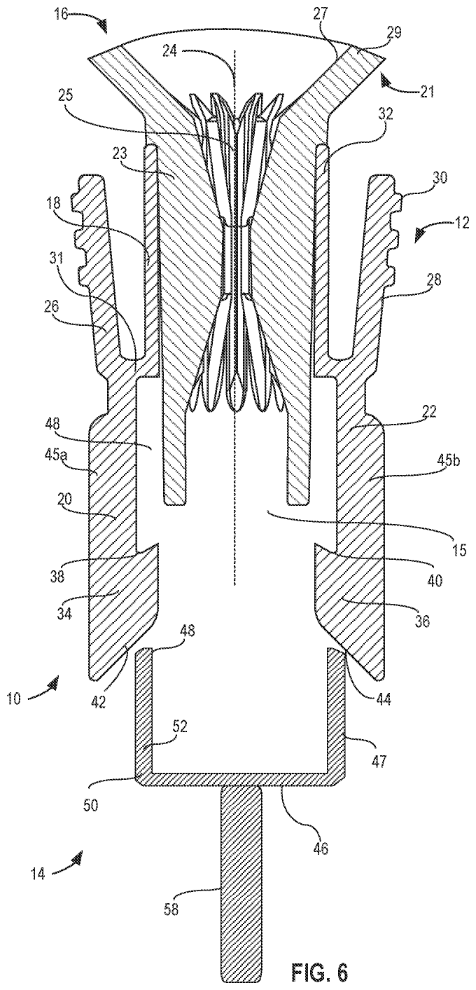

Various embodiments of the present disclosure may include dust caps 14 configured for covering the nozzle of the syringe 16, such as described herein, for example, with reference to FIGS. 6-8. The dust cap 14 may be configured to maintain the sterility of the sterile fluid path associated with the syringe 16 and the connector element 12. As used herein, the term “sterile fluid path” means the components, portions, or surfaces of the syringe 16 and/or associated fluid path elements, such as the connector element 14, that may come into contact with a medical fluid that is injected into a patient and are required to be sterile for the safety of the patient. The sterile fluid path may be sterilized, for example by a conventional sterilization procedure such as electron beam or ethylene oxide exposure, during a manufacturing or packaging process, and the various components and portions of the sterile fluid path may remain sterile even if other portions of the syringe 16, such as the outer surface of the barrel, are exposed to an unsterilized environment. Thus, the sterile fluid path approach does not require packaging features that are impenetrable to environmental and microbial contaminants, but the various sterile surfaces of the sterile fluid path are not exposed to environmental or microbial contaminants.

With reference to FIGS. 1-4, syringe 16 has a barrel 17 with a proximal end 19 and a distal end 21 having a discharge nozzle 23. In some examples of the present disclosure, the fluid path connector assembly 10, such as the connector element 12 thereof, is configured for removably connecting to the discharge nozzle 23 of syringe 16. In further examples, a first portion of the fluid path connector assembly 10, such as the connector element 14, may be non-removably connected to the discharge nozzle 23 of syringe 16, while the dust cap 14 is removably connected to the connector element 14. Syringe 16 may be any syringe known in the art, for example, a bottle or a rolling diaphragm syringe as disclosed in WO2016/172467, WO2015/164783, and WO2016/069711, incorporated by reference herein. In other embodiments, the dust caps described herein may be used on an outlet of a peristaltic pump.

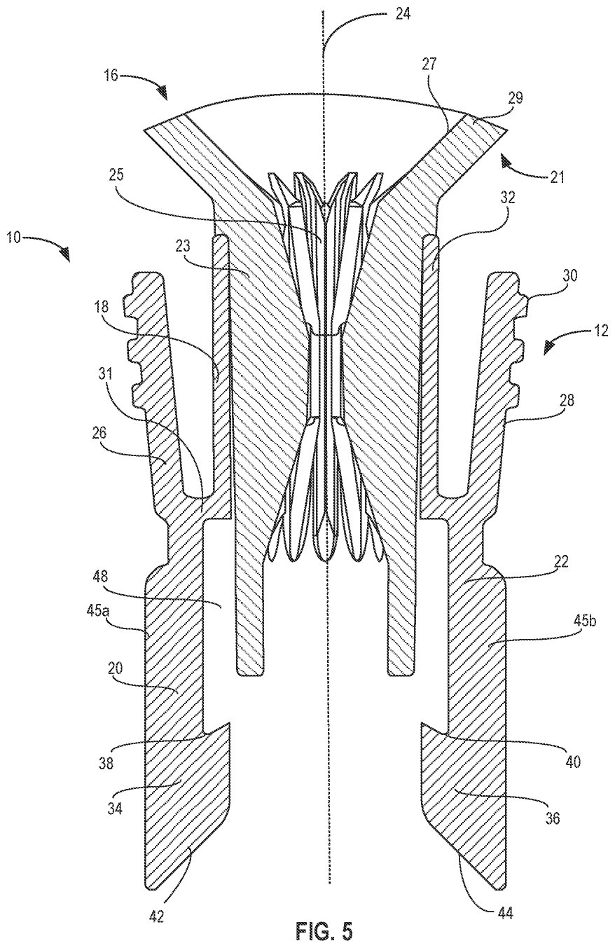

With reference to FIG. 5, in one example of the present disclosure, an inner surface of a distal end 21 of the syringe 16 may include a plurality of ribs 25 that create a contour in the syringe 16 that takes advantage of a Coanda effect for the fluid passing through the syringe 16. As used herein, the Coanda effect is the tendency for a liquid stream to be attracted to a nearby curved or angled surface as the liquid flows along the surface. Thus, as fluid enters the syringe 16 through the connector element 14, the fluid is diverted towards the inner walls of the discharge nozzle 23 of the syringe 16 to contact the plurality of ribs 25. As fluid flows along the ribs 17, the fluid is naturally attracted to the inside surface of the conical distal end 21 of the syringe 16. The fluid then flows down an inner surface 27 of a tubular sidewall 29 of the syringe 16, ultimately accumulating at the bottom of the syringe 16, filling syringe 16 from the bottom up as air escapes the syringe 16 through the distal end 21. This flow along the inside surface of the syringe 16 helps to reduce turbulence as the fluid fills the syringe 16, which aides in reducing air bubbles from forming as the syringe 16 is filled. Further, the ribs 25 allow for more rapid filling of the syringe 16, thereby reducing the duration of the fluid injection process. Further features and advantages of this Coanda effect are described in WO2017/091643, the disclosure of which is incorporated by reference in its entirety.

With reference to FIGS. 1-5, the connector element 12 may be operatively connected to a distal end 21 of the syringe 16. In one example of the present disclosure, the connector element 12 may be welded (for example, laser welded), chemically welded, or otherwise adhered to the distal end 21 of the syringe 16.

With continued reference to FIGS. 1-5, in some examples of the present disclosure, the connector element 12 may include a body 18, a lumen 15 (shown in FIG. 5) extending through the body 18, a first flexible leg 20 (hereinafter referred to as “first flexible leg” or “first leg”), and a second flexible leg 22 (hereinafter referred to as “second flexible leg” or “second leg”) each having a connection beam 31 (shown in FIG. 5) connecting the first flexible leg 20 and the second flexible leg 22 to the body 18. The first and second flexible legs 20, 22 may be formed integral with the body 18. The first and second flexible legs 20, 22 may extend from the body 18 in a distal direction relative to the distal end 23 of the syringe 16 as shown in FIGS. 1-5. In one example of the present disclosure, the first and second flexible legs 20, 22 are made of a material such that they can pivot where the flexible legs 20, 22 meet the connection beam 31 to allow the distal end of the first and second flexible legs 20, 22 to move radially outward in a direction relative to a longitudinal axis 24 of the connector element 12. In one example of the present disclosure, the first and second flexible legs 20, 22 may be made of a rigid material with a flexible pivot point to allow the first and second flexible legs 20, 22 to move radially outward relative to a longitudinal axis 24 (shown in FIG. 5) of connector element 12.

In some examples of the present disclosure, the connector element 12 may also include a first actuating arm 26 and a second actuating arm 28 that extend from the body 18 in a direction opposite to the first and second flexible legs 20, 22. The first and second actuating arms 26, 28 may be formed integral with the body 18 and the first and second flexible legs 20, 22. In some examples, the first and second flexible legs 20, 22 and the first and second actuating arms 26, 28 may be connected to the body 18 of the connector element 12 via the connection beam 31. During operation of the fluid path connector assembly 10, the first and second actuating arms 26, 28 are configured to be pushed radially inwards towards one another by an operator to move the first and second flexible legs 20, 22 radially outwardly away from one another. Pressure on the first and second actuating arms 26, 28 forces the first and second flexible legs 20, 22 to move away from one another. In one embodiment of the present disclosure, at least one of the first and second actuating arms 26, 28 may include a plurality of gripping ribs 30 to assist in gripping the first and second actuating arms 26, 28 when handling the connector element 12. In some examples of the present disclosure, the connector element 12 may also include a support portion 32 that extends from the body 18 in the same direction as the first and second actuating arms 26, 28, that is, toward the body of the syringe 16 as shown in FIGS. 1-6. The support portion 32 may be formed integral with the body 18 and may define a channel configured to receive the distal end 23 of the syringe 16. The support portion 32 may be connected to the distal end of the syringe 16, for example by a screw fit, adhesion, or welding. The support portion 32 provides a stabilizing effect to the connector element 12 when the connector element 12 is operatively connected to syringe 16 and, further, when first and second actuating arms 26, 28 are pressed inwardly towards one another to actuate connector element 12.

According to various examples of the present disclosure, each of the first and second flexible legs 20, 22 may include a first flange 34 and a second flange 36, respectively, that are configured to engage with a portion of the dust cap 14, as described herein, to readily connect the connector element 12 with the dust cap 14. In some examples of the present disclosure, at least a portion 38, 40 of each flange 34, 36, respectively, may be angled inwardly relative to the longitudinal axis 24 of the connector element 12. In one example of the present disclosure, the portions 38, 40 of the flanges 34, 36 may be pointed towards the distal end 21 of the syringe 16 such that the portions 38, 40 of the flanges 34, 36 extend towards a proximal end of the syringe 16. Under fluid injection pressures, the angled portions 38, 40 of the flanges 34, 36 may force the first and second legs 20, 22 in a radially inward direction, thereby increasing the strength of the connection force between the connector element 12 and the dust cap 14 during a pressurized injection and making disconnection unlikely. The distalmost surface 42, 44 of the flanges 34, 36 may be sloped or beveled to assist in receiving the dust cap 14, as described below. The surfaces 42, 44 may be angled inwardly towards the longitudinal axis 24 of the connector element 12. In one example of the present disclosure, an outer edge of each surface 42, 44 may slope inwardly to an inner edge of each surface 42, 44 while sloping inwardly and in a direction towards the actuating arms 26, 28. The beveled or sloped surfaces 42, 44 flex the first and second flexible legs 20, 22 radially outward during insertion of the dust cap 14 into the connector element 12 and then the first and second flexible legs 20, 22 snap back in a radially inward direction once the body 46 of the dust cap 14 passes the flanges 34, 36, thereby engaging the connector element 12 with the dust cap 14. The snapping back action may produce an audible click to provide confirmation that dust cap 14 is properly connected to connector element 12.

According to various examples of the present disclosure, each of the legs 20, 22 may also include at least one reinforcing member 45a, 45b. According to various examples, the reinforcing members 45a, 45b may be provided along at least a portion of an outer surface of the legs 20, 22. In certain examples, the reinforcing members 45a, 45b may extend the entire length of the legs 20, 22. The reinforcing members 45a, 45b may be formed integral with the legs 20, 22, for example on an outer surface of the leg 20 or 22. The reinforcing members 45a, 45b may be made of a similar material as the legs 20, 22 and the connector element 12, for example, by co-molding the leg 20, 22 with the respective reinforcing member 45a, 45b. In other examples, the reinforcing members 45a, 45b may be made of a different material than the legs 20, 22 and the connector element 12 to increase the strength of the legs 20, 22. The reinforcing members 45a, 45b may be provided on the legs 20, 22 to add rigidity to the legs 20, 22 while still allowing the legs 20, 22 to retain sufficient flexibility to move inwardly and outwardly during connection and disconnection of the connector element 12 and the dust cap 14. In one example of the present disclosure, the reinforcing members 45a, 45b may be strips of material that extend along at least a portion of the length of the legs 20, 22 and extend outwardly from an outer surface of the legs 20, 22, for example, substantially perpendicular to the outer surface of the legs 20, 22. After the dust cap 14 is connected to the connector element 12, the reinforcing members 45a, 45b may be configured to assist in maintaining the connection of the legs 20, 22 to the dust cap 14, for example by further reducing outward flexing or stretching of the legs 20, 22.

With reference to FIGS. 6-8, according to some examples of the present disclosure, the dust cap 14 for ensuring non-contamination through the discharge nozzle 23 of syringe 16 is described in detail. The dust cap 14 may be configured to readily engage with the distal end 21 of the syringe 16 and be removably retained on the discharge nozzle 23 by the connector element 12 to maintain the sterility of the lumen of the connector element 12 and the interior volume of the syringe 16. According to various embodiments, the dust cap 14 may be configured to be readily removed from the distal end 21 of the syringe 16 prior to connection of one or more fluid path elements with the connector element 12. During the setup of one or more fluid delivery procedures prior to an injection of a contrast and/or saline, it is critical that the setup be performed quickly and accurately so the overall time if the imaging procedure is reduced, thereby allowing for a greater number of imaging procedures being performed in a day. This allows for a higher number of patients treated. It is also important that the setup procedure be performed accurately to ensure that the fluid injector is ready for delivering fluid to the patient. One step in the fluid injector setup process is removal of the dust cap and attachment of a fluid line to fill the syringe with fluid and/or deliver the fluid to the patient, while maintaining the sterility of the fluid path. Various embodiments of the present disclosure provide for dust cap features that allow for rapid removal while maintaining sterility of the fluid path. In addition, the dust cap designs herein may further include one or more tamper evident or tamper resistant features to prevent tampering with the dust cap and ensuring sterility of the fluid path and/or notifying medical personnel that the syringe/dust cap assembly may have been tampered with or the syringe previously used, thereby impacting the sterility of the system and potential danger to the patient.

In certain embodiments, the dust cap 14 may be configured to be readily removed by medical personnel grasping a tab element 58 extending distally from the dust cap 14, for example by simply pulling the dust cap 14 in the distal direction to remove the dust cap from the distal 14 connector element 12. For example, while the dust cap 14 is configured to engage the connector element 12 and the flexible legs 20, 22 while maintaining the sterility of the fluid path of syringe 16, the dust cap 14 may be removed without the medical personnel radially displacing of the flexible legs 20, 22 to release the dust cap. In some examples, the dust cap 14 is configured to be removed from the connector element 12 with one hand, for example after the syringe 16 has been installed on the fluid injector. The dust cap 14 may be made from a rigid, medical grade material that is substantially resistant to deflection or deformation. In some examples, the dust cap 14 is made from a flexible material that is compliant to deflection.

The dust cap 14 may include a body 46 configured for connection to the connector element 12, for example by a perimeter skirt 47. In some examples of the present disclosure, the perimeter skirt 47 of the body 46 may be configured to substantially surround the outer surface of the body of the connector element 12 to enclose the lumen 15. In other embodiments the perimeter skirt 47 of the body 46 may have a smaller diameter than the body 18 of the connector element 12 and be configured to substantially fit within and engage the inner surface of the body 18 of the connector element 12 to enclose the lumen 15. The body 46 may be substantially cylindrical in shape. Upon connection of the dust cap 14 with the connector element 12, the body 46 may be configured to be removably held between the legs 20, 22 of the connector element 12. The body 46 may assist in maintaining sterility of the fluid path, for example by preventing inadvertent touching and contamination of the body of the connector element 12 during manipulation of the syringe 16 and the fluid path connector assembly 10 and/or by preventing ingress of contaminants into the sterile fluid path of the syringe 16. In some examples, the dust cap body 46 may be rotatable relative to the body 18 of the connector element 12 about the longitudinal axis 24. In further examples, the dust cap body 46 may be fixed from rotation relative to body 18 of the connector element 12 about longitudinal axis 24.

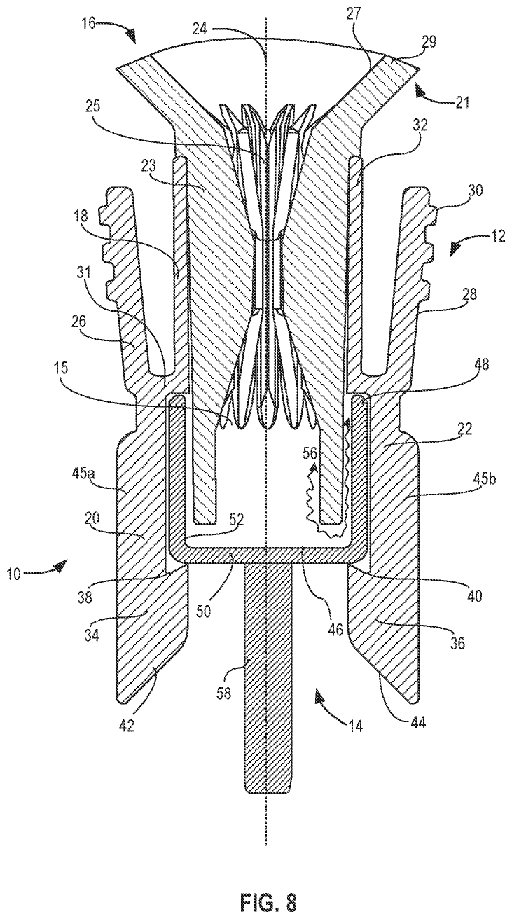

With reference to FIG. 6, the dust cap body 46 has a proximal end 48 opposite a distal end 50 with a sidewall 52 extending between the proximal end 48 and the distal end 50. The sidewall 52 may be substantially cylindrical. When connected to the connector element 12, the dust cap body 46 is configured to extend from the connection beam 31 at the proximal end 48 and engage the distal end 50 of the dust cap 14 with the flange portions 38, 40 of flexible legs 20, 22. The proximal end 48 may be open and be configured to extend over at least a portion of the body 18 of the connector element 12 so as to enclose the lumen 15 (shown in FIG. 5). The distal end 50 is closed to prevent entry of contaminants into an interior 54 of the dust cap body 46. As shown in FIG. 8, when the dust cap 14 is connected to the connector element 12, a tortuous fluid path 56 is defined between the dust cap body 46 and the body 18 of the connector element 12 to permit equalization of air pressure with the interior volume of the syringe 16 through the lumen 15 of the connector element 12 while preventing a passage of contaminants into the lumen 15 of the connector element 12 and into the interior of syringe 16. In this manner, air can freely pass through the lumen 15 of the connector element 12 such that no pressure can be built up within the interior of the syringe 16 due to temperature or atmospheric pressure changes during shipping and storage. For example, during shipping (e.g., by air) or storage (at different temperatures), the syringe may experience changes in atmospheric pressure producing a pressure gradient between the interior volume and exterior of the syringe 16, which may result in the dust cap 14 or syringe plunger (not shown) being ejected off or out of the syringe 16. By equalizing the pressure to remove the pressure gradient, dust cap 14 or plunger ejection may be avoided.

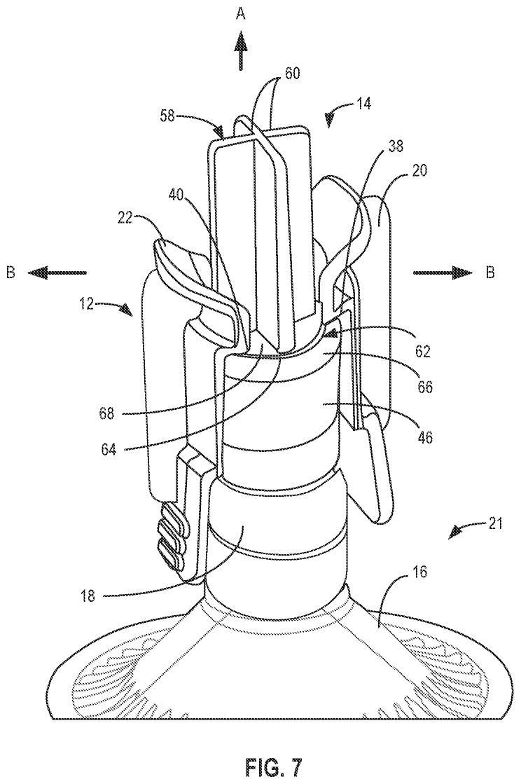

With reference to FIGS. 6-8, the dust cap 14 may include a grasping tab 58 connected to the distal end 50 of the dust cap body 46. In some examples, the grasping tab 58 has one or more gripping elements 60 configured to facilitate gripping and removal of the dust cap 14 from the connector element 12. For example, the one or more gripping elements 60 may be arranged in an X-shaped configuration shown in FIG. 7. In other embodiments, the gripping elements 60 may include one or more lateral bumps along the surface of grasping tab 58.

With reference to FIG. 7, the dust cap 14 further includes an engagement element such as ledge 68 at the distal end 50 (shown in FIG. 6) of the dust cap body 46. When the dust cap 14 is connected to the connector element 12, the engagement element 62 is configured to be in contact with portion 38 of first flange 34 of the first flexible leg 20 and portion 40 of second flange 36 of the second flexible leg 22 of the connector element 12 (shown in FIG. 8) to prevent disengagement of the dust cap 14 from the connector element 12. In this manner, the dust cap 14 may be maintained on the connector element 12 to ensure sterility of the fluid path of syringe 16. In various embodiments herein, dust cap 14 may be quickly removed by the medical personnel during the fluid path assembly step without need for applying pressure to radially displace the flexible legs 20, 22. For example, according to various embodiments, the medical personnel may simply grasp grasping tab 58 and pull the dust cap 14 in a distal direction to remove the dust cap 14 from the connector element 12, saving additional time during a setup process. During the manufacturing, shipping, and unpackaging processes, the dust cap 14 is retained by one or more of a friction fit with at least a portion of the connector element 12 and a retention force between the distal end 50 of the dust cap 14 and the flanges 34, 36 of the flexible legs 20, 22.

According to various examples, engagement element 62 has a sloping member 64 defined by a distal edge 66 of the dust cap body 46. The sloping member 64 extends around at least a portion of an outer perimeter of the distal edge 66 of the dust cap body 46. In some examples of the present disclosure, the sloping member 64 is a rounded circumferential edge or a chamfered circumferential edge. When the dust cap 14 is connected to the connector element 12 and is being removed by medical personnel, the first flange 34 of the first flexible leg 20 and the second flange 36 of the second flexible leg 22 of the connector element 12 are configured to contact the sloping member 64 when the dust cap 14 is moved distally relative to the connector element 14 in a direction of arrow A in FIG. 7 such that the first flexible leg 20 and the second flexible leg 22 expand radially outward in a direction of arrows B in FIG. 7 to permit removal of the dust cap 14 from the connector element 14. The engagement element 62 further includes a ledge 68 configured to contact the first flange 34 of the first flexible leg 20 and the second flange 36 of the second flexible leg 22 of the connector element 12 when the dust cap 14 is connected to the connector element 12 to retain the dust cap 14 on the connector element 14 when not needing to be removed. The ledge 68 may be oriented in a direction substantially perpendicular to a direction of the longitudinal axis 24.

The dust cap 14 according to any of the various configurations described herein may further include a tamper resistant and/or tamper evident feature 70 to inform medical personnel or user that the sterility of the sterile fluid path may be compromised, for example due to damage during manufacturing, packaging, shipping, or unpackaging or that the syringe 16 may have been previously used or tampered with and is no longer sterile. As the syringe 16 is being used to deliver a medical fluid to a patient's arterial or venous system, for the safety of the patient it is critical that interior surfaces of the syringe 16 and associated fluid path that contact the medical fluid be sterile and non-contaminated with microbial contamination, inadvertent or intentional contact with a hazardous compound, or bodily fluids from a previous patient.

The tamper resistant and/or tamper evident feature 70 may be removed or disengaged upon unpackaging or after the syringe 16 has been installed onto an injector. The tamper resistant and/or tamper evident feature 70 according to various embodiments may be configured to work in conjunction with the dust cap 14 to ensure the sterility of the fluid path of the syringe 16 and associated fluid path elements. Once the tamper resistant and/or tamper evident feature 70 is removed, the medical personnel may then grasp the grasping tab 58 of the dust cap 14 and remove the dust cap 14 from the connector element 12 and attach one or more fluid path components to the connector element 12, such as tubing for delivering fluid from the syringe 16 to a patient. According to various examples, damage or change to the tamper resistant and/or tamper evident feature 70 informs the medical personnel that the syringe 16 may no longer be sterile or safe to use and should be replaced with a new, sterile syringe. When present, the tamper resistant and/or tamper evident feature 70 is configured to prevent early removal of the dust cap 14 from the connector element 12 and to also help maintain engagement between the dust cap 14 and syringe 16 during manufacture, shipping, storage, and setup. Removal of the tamper resistant and/or tamper evident feature 70 is configured to permit removal of the dust cap 14 from the connector element 12.

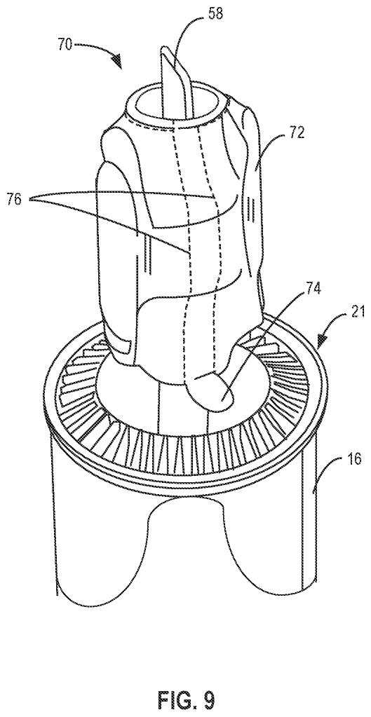

With reference to FIG. 9, and in accordance with one example, the tamper resistant and/or tamper evident feature 70 is a seal 72 that covers at least a portion of the connector element 12 and the dust cap 14 to at least partially enclose the connector element 12 and the dust cap 14 and keep the dust cap 14 engaged with the connector element 12. In some examples, the seal 72 may be a shrink-wrapped seal made from a heat-shrinkable plastic material or other shrinkable plastic material (e.g., solvent shrinkable) that at least partially conforms to the shape of the connector element 12 and the dust cap 14. When present, the seal 72 is configured to prevent removal of the dust cap 14 from the connector element 12 and to show that the dust cap 14 has not been previously removed from the syringe 16, thus ensuring sterility of the components. The seal 72 may have one or more removal tabs 74 that allows for ready removal of the seal 72 from the connector element 12 and the dust cap 14. The removal tab 74 may be connected to the seal 72 by at least one tearable perforated strip 76 in such a way that pulling the removal tab 74 away from the seal 72 years the at least one tearable perforated strip 76. In some examples, a plurality of tearable perforated strips 76 may be spaced apart radially around the connector element 12 and the dust cap 14. The plurality of tearable perforated strips 76 may be spaced apart from each other at equal or unequal intervals. In other embodiments, the seal 72 may include at least one tearable perforated strip 76 having one or more starter notches at the edge of the seal 72 that initiate tearing of the tearable strip. In certain embodiments, the at least one tearable strip may include a reinforced non-tearable portion embedded into or otherwise attached along the strip to ensure complete tearing along the longitudinal axis of the strip 76. The removal tab 74 may be provided on a proximal or distal portion of the seal 72, and may be removed by a pulling movement in a distal or proximal direction, respectively. In further examples, a pair of removal tabs 74 may be provided such that the tearable perforated strip 76 can be removed by pulling either in the proximal or distal direction. In some examples, the tearable perforated strip 76 may be linear, such as shown in FIG. 9. In further examples, the removal tab 74 may extend in a diagonal direction relative to the longitudinal axis 24 (shown in FIG. 5), or in a helical direction around the longitudinal axis 24 such that the removal tab 74 is removed via a rotational movement about the longitudinal axis 24.

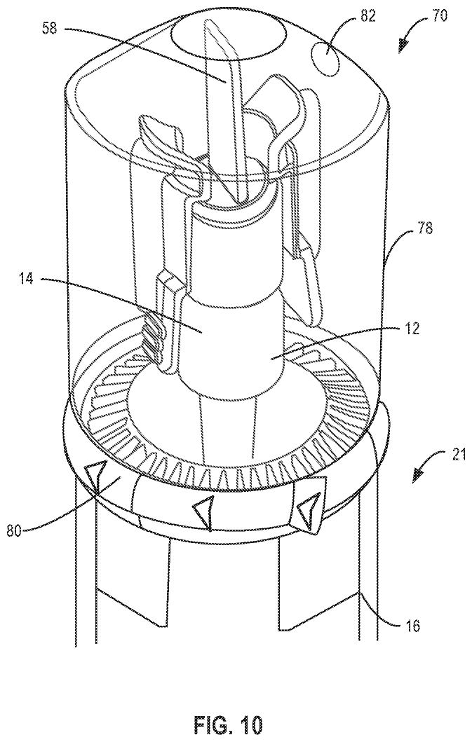

With reference to FIG. 10, an example of the tamper resistant and/or tamper evident feature 70 may include a cap 78, such as a plastic cap, at least partially enclosing the connector element 12 and the dust cap 14. In certain embodiments, the cap 78 may cover the entire distal end 21 of the syringe 16, including the entirety of the connector element 12 and the dust cap 14. Alternatively, the cap 78 may only cover a portion of the distal end 21 of the syringe 16, including a portion of the connector element 12 and the dust cap 14. For example, the protective cap may include one or more portions that extend over the distal grasping tab 58 of the dust cap 14 and retain the dust cap 14 connected to the connector element 12. The cap 78 may be attached to the distal end of the syringe 16 by a snap fit or friction fit.

The cap 78 may be at least partially transparent such that the connector element 12 and/or the dust cap 14 can be visually observed through the sidewall of the cap 78, such that medical personnel may see the connector element 12 and dust cap 14 to ensure that they remain engaged. In some examples, the cap 78 may be opaque. In some examples, the cap 78 is made from a medical grade plastic material. The cap 78 may alternatively be made from another medical grade material that prevents access to and/or removal of the dust cap 14 prior to use. For example in certain examples, the cap 78 may be made of a paper, cardstock, cardboard, or other readily recyclable material.

With continued reference to FIG. 10, a cap 78 may have at least one vent 82 that allows for an exchange of air and pressure equalization between an interior of the cap (and ultimately the interior of the syringe via the dust cap 14) and the outside atmosphere to prevent an air pressure gradient or temperature-related buildup of pressure in the syringe 16 during shipping and storage, as described herein.

The cap 78 may be secured to the syringe 16, such as the distal end 21 of the syringe 16, via a connection element, such as a tearable strip or adhesive 80. In certain examples, the tearable strip 80 may seal a proximal end of the cap 78 with distal lip of the circumferential barrel of the syringe. In some examples, the cap 78 may be configured for a push-fit or snap fit connection with a circumferential edge 84 (shown in FIGS. 1-4) at the distal end 21 of the syringe 16.

To prepare the syringe 16 for use, the medical personnel may remove the tearable or adhesive strip 80 from the cap 78 and remove the cap 78 from the syringe 16 by pulling the cap 78 in a distal direction or by applying a torque to the cap 78. In some examples, an inner portion of the cap 78 may be connected to the dust cap 14, such as the grasping tab 58. In this manner, removal of the cap 78 from the syringe 16 also removes the dust cap 14 from the connector 12. Once removed, the cap 78 may be disposed or recycled. In some examples, the removed cap 78 can be used for collecting a volume of prime fluid during a priming operation of a fluid injector. In some examples, the cap 78 may be made from a breakable plastic material such that the cap 78 is configured to break upon removal from the syringe 16 to prevent reconnection and reuse of the cap 78 with the syringe 16 and to ensure that the syringe 16 and cap 78 are not reused.

With reference to FIG. 11, and according to another example, a tamper resistant and/or tamper evident feature 70 may include a reinforcing band 86 that surrounds an outer perimeter of at least a portion of the first and second flexible legs 20, 22. The reinforcing band 86 may be made from paper, other textile material, and/or plastic, or combinations thereof. While plastic and/or paper are suitable materials for the reinforcing band 86, other materials that may wrap around the flexible legs 20, 22 are also contemplated. The presence of the reinforcing band 86 around the first and second flexible legs 20, 22 prevents movement of the flexible legs 20, 22 in a radially outward direction. In this manner, if removal of the dust cap 14 is attempted while the reinforcing band 86 is connected, the reinforcing band 86 prevents the deflection of the first and second flexible legs 20, 22 and thereby prevents the removal of the dust cap 14. In further examples, removal of the dust cap 14 may break the reinforcing band 86, thereby providing an indication that the connector element 12 may have been tampered with or previously used. Tampering with, potential contamination of, or previous use of the syringe 16 may be indicated by the absence of or damage to the reinforcing band 86.

In some examples, opposing ends of the reinforcing band 86 may be adhesively, heat sealed, or otherwise secured to one another to surround at least a portion of the first and second flexible legs 20, 22. At a site 88 of connection between the opposing ends, the reinforcing band 86 may include a pull tab 90 that can be readily grasped and pulled by the medical personnel to disconnect the opposing ends and remove the reinforcing band 86. As the reinforcing band is removed, the flexible legs 20, 22 may then freely move in a radially outward direction as the dust cap 14 is removed from the connector element 12, as described herein.

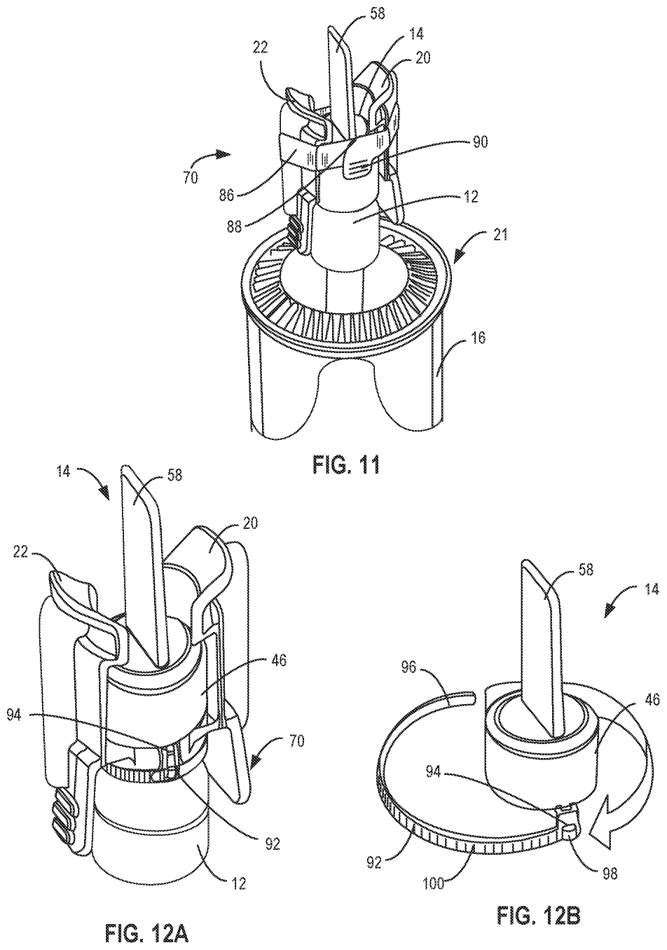

With reference to FIGS. 12A-12B, and according to another example, a tamper resistant and/or tamper evident feature 70 may include a band 92 connected to the connector element 12 and a breakable connecting tie 94 having a first end connected to the band 92 and a second end connected to the dust cap 14. In some examples, the band 92 and the breakable connecting tie 94 may be integrally formed with dust cap 14, such as being molded therewith.

In some examples, such as shown in FIG. 12B, the band 92 may be a zip tie having a first free end 96 connectable with a connector 98 on a second end 100. The zip tie may be configured to wrap around a perimeter of the connector element 12, for example proximal to connection beams 31 which connect the flexible legs 20, 22 with the body 18 of the connector element 12, such that the first free end 96 can be inserted through the connector 98 of the second end 100 to secure the zip tie to the connector element 12. In this way, the dust cap 14 can be securely connected with the connector element 12, with the band 92 and the breakable connecting tie 94 preventing removal of the dust cap 14 without disconnection of the tamper resistant and/or tamper evident feature 70, for example by breaking the breakable connecting tie 94 from the dust cap 14. In further examples, the band 92 may be integrally formed with the connector element 12, or the band 92 can be connected to the connector element 12, such as by welding, during manufacture.

In other embodiments, the band 92 may include an adhesive connection connecting the first free end 96 with the second end 100, wherein the adhesive connection may be readily broken during removal of the dust cap 14. According to these embodiments, the band 92 may be made from a polymeric, plastic material integrally formed with the dust cap 14. Alternatively, the band 92 may be made from a paper textile type material that may be adhered to the dust cap 14, for example where the second end 100 at the breakable connecting tie 94. Securing band 92 around the circumference of the connector element 12 prevents inadvertent removal of dust cap 14 from connector element 12 unless band 92 is first disengaged.

The breakable connecting tie 94 may be broken by grasping the grasping tab 54 of the dust cap 14 and pulling the grasping tab 58 in a distal direction A along the longitudinal axis 24 (shown in FIG. 7) and/or rotating the grasping tab 58 about the longitudinal axis 24 to break the connection between the breakable connecting tie 94 and the dust cap body 46. Tampering with, potential contamination of, or previous use of the syringe 16 may be indicated by the absence of or damage to the breakable connecting tie 94. In certain embodiments, the band 92 may be disengaged by disconnecting the first end 96 from the second end 98, for example when the first end 96 is adhesively connected to the second end 98, allowing removal of the dust cap 14.

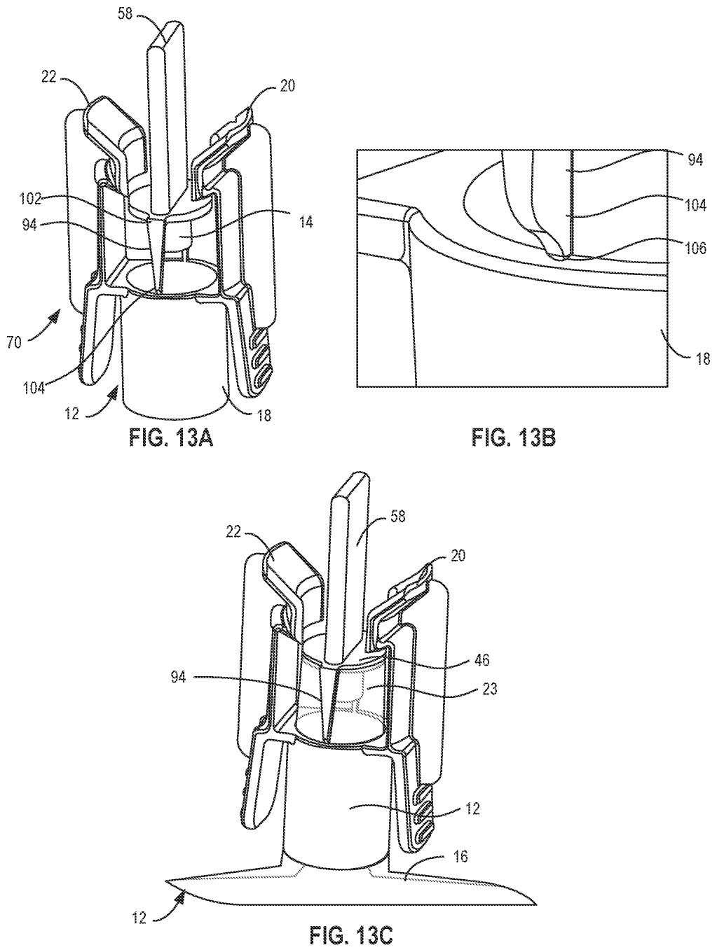

With reference to FIGS. 13A-13C according to another example, the breakable connecting tie 94 has a first end 102 connected to dust cap 14 and a second end 104 in the form of a point attachment integrally formed with and connected to the connector element 12. In some examples, breakable connecting tie 94 may be formed monolithically with the connector element 12 and the dust cap 14, such as via an injection molding process. The second end 104 may have a weakened portion 106 (shown in FIG. 13B), such as the point attachment, that is configured to break, disconnect, or tear when the grasping tab 54 of dust cap 14 is pulled in a distal direction along longitudinal axis 24 and/or rotated about longitudinal axis 24 (FIG. 8).

When the connector element 12 is connected to the syringe 16, such as shown in FIG. 13C, the dust cap body 46 is arranged to sealingly fit within the discharge nozzle 23 at the distal end 21 of the syringe 16. In some examples, the dust cap body 46 may surround the discharge nozzle 23 of the syringe 16. In this manner, the sterile fluid path of the syringe interior may be maintained while the dust cap 14 is in place. The dust cap 14 may be removed when the grasping tab 54 of the dust cap 14 is pulled in a distal direction along the longitudinal axis 24 (shown in FIG. 8) and/or rotated about the longitudinal axis 24, which breaks the breakable connecting tie 94 at the weakened portion 106. When the dust cap 14 is removed, the syringe 16 may be attached to the various fluid path elements during setup for the fluid injection procedure. Tampering with, potential contamination of, or previous use of the syringe 16 may be indicated by the disconnection of the breakable connecting tie 94 from connector element 12.

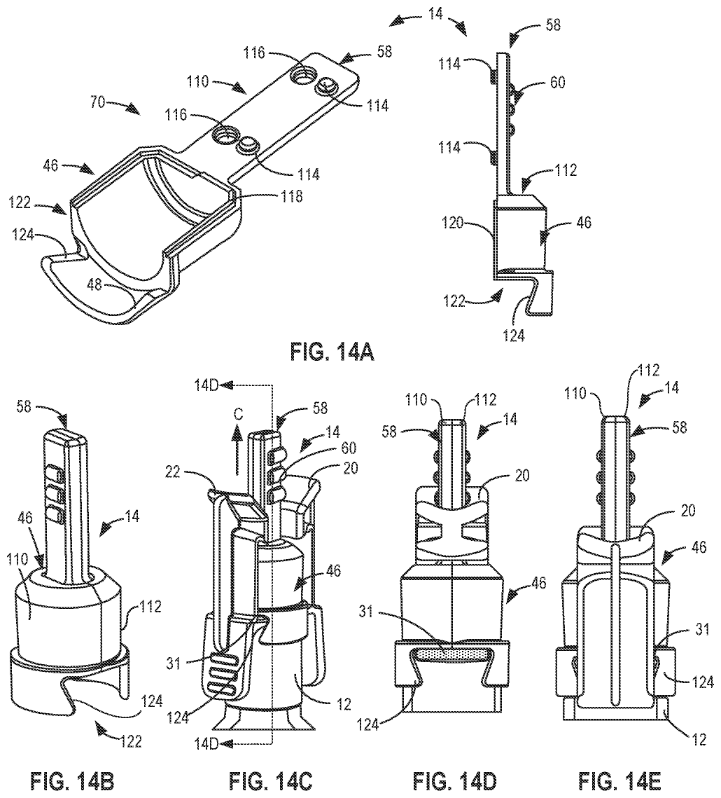

With reference to FIGS. 14A-14E, and according to another example, a tamper resistant and/or tamper evident feature 70 may be embodied as a two-piece dust cap 14. With reference to FIGS. 14A-14B, the dust cap 14 has a first portion 110 that is removably connectable to a second portion 112. In some examples, the first portion 110 and the second portion 112 may be joined together on a plane that is substantially parallel with a direction of the longitudinal axis 24 (shown in FIG. 14C). The first portion 110 and the second portion 112 may have one or more alignment bosses 114 and corresponding one or more alignment indents 116 that help align the first portion 110 with the second portion 112 during connection to form the dust cap 14. The one or more alignment bosses 114 are configured to align with and engage the opposing one or more alignment indents 116, for example by a snap fit, to correctly orient the first and second portions 110, 112 of the two-piece dust cap 14. In some examples, the one or more alignment bosses 114 and corresponding one or more alignment indents 116 may be formed on inner surfaces of the first portion 110 and the second portion 112 facing each other along the grasping tab 58. The one or more alignment bosses 114 may be one or more protrusions on one of the first portion 110 and the second portion 112 that are configured to connect with one or more alignment indents 116 on the other of the first portion 110 and the second portion 112.

In one configuration, the first portion 110 and the second portion 112 may be identical such that the one or more alignment bosses 114 on one side of the grasping piece of the first portion 110 engage the corresponding one or more alignment indents 116 on the other side of the grasping tab piece of the second portion 112. In another configuration the first portion 110 may include only the one or more alignment bosses 114 and the second portion 112 may include only the one or more alignment indents 116. Other arrangements and configurations of the one or more alignment bosses 114 and the one or more alignment indents 116 are also considered.

The one or more alignment bosses 114 and corresponding one or more alignment indents 116 may be formed on, for example, the grasping tab 58 of the dust cap 14. The first and second portions 110, 112 may be joined together via a frictional or snap fit between the one or more alignment bosses 114 and corresponding one or more alignment indents 116, and/or via other means, such as using an adhesive or plastic welding.

With continued reference to FIG. 14A, the dust cap 14 may further include at least one tongue 118 and a corresponding at least one groove 120 configured to connect with the at least one tongue 118. In some examples, the at least one tongue 118 and the at least one groove 120 may be provided on the dust cap body 46 and are configured to align and hold the first and second portions 110, 112 of the two-piece dust cap 14 in an engaged orientation. The at least one tongue 118 and the at least one groove 120 may be provided on opposite sides of the first portion 110 and on opposite sides of the second portion 112 in a configuration such that the tongue 118 on the first portion 110 engages a corresponding groove 120 on the second portion 112 and the tongue 118 on the second portion 112 engages a corresponding groove 120 on the first portion 110. In other embodiments, the first portion 110 may include the at least one tongue 118 and the second portion 112 may include the at least one groove 120 in a configuration such that tongue 118 on first portion 110 engages corresponding groove 120 on second portion 112.

With continued reference to FIGS. 14A-14B, the proximal end 48 of each side of the first portion 110 and the second portion 112 of the two-piece dust cap 14 may include an undercut 122 extending through the sidewall of the dust cap body 46. In some examples, the undercut 122 may be configured to aid in separation of the first portion 110 from the second portion 112 during removal of the dust cap 14 from the connector element 12 (shown in FIGS. 14C-14E). The undercut 122 may include at least one tapered or angled lateral surface 124 that is configured to interact with at least a portion of the connector element 12, for example, the connection beam 31 that connects the body 18 of the connector element 12 to the first and second flexible legs 21, 22. In this manner, during a removal process, such as by distally pulling on the grasping tab 58, the at least one angled lateral surface 124 acts as a wedge to separate the first and second portions 110, 112 of the dust cap 14. In particular, the at least one angled lateral surface 124 of the undercut 122 is configured to slide along connection beam 31 of the connector element 12 (shown in FIG. 14D) as the dust cap 14 is moved in the direction of arrow C in FIG. 14C, which forces the proximal ends 48 of the two-piece dust cap 12 in a radially outward direction, thereby disengaging the least one tongue 118 and the at least one groove 120 and/or the one or more alignment bosses 114 and the one or more alignment indents 116 of the first and second portions 110, 112 of the dust cap 14 during removal.

The two-piece dust cap 14 may be assembled on the connector element 12 and aligned so that the one or more alignment bosses and tongue and groove alignment features of one of the first portion 110 and the second portion 112 engage with the complementary features on the other of the first portion 110 and the second portion 112. The first and second portions 110, 112 may then be adhesively connected, for example using a UV process or other adhesion process or by a snap fit. Once adhered to each other, the first and second portions 110, 112 are breakably connected with each other, such as due to engagement of the angled lateral surface 124 of the undercut 122 with the connection beam 31 of the connector element 12 as the dust cap 14 is moved in the direction of arrow C in FIG. 14C. Once removed, the dust cap 14 is broken into two pieces (i.e., the first portion 110 and the second portion 112), which are configured to prevent reassembly. Tampering with, potential contamination of, or previous use of the syringe 16 may be indicated by observations of at least partial disconnection of the first and second portions 110, 112 of the two-piece dust cap 14 or absence of the dust cap 14 from the connector element 12.

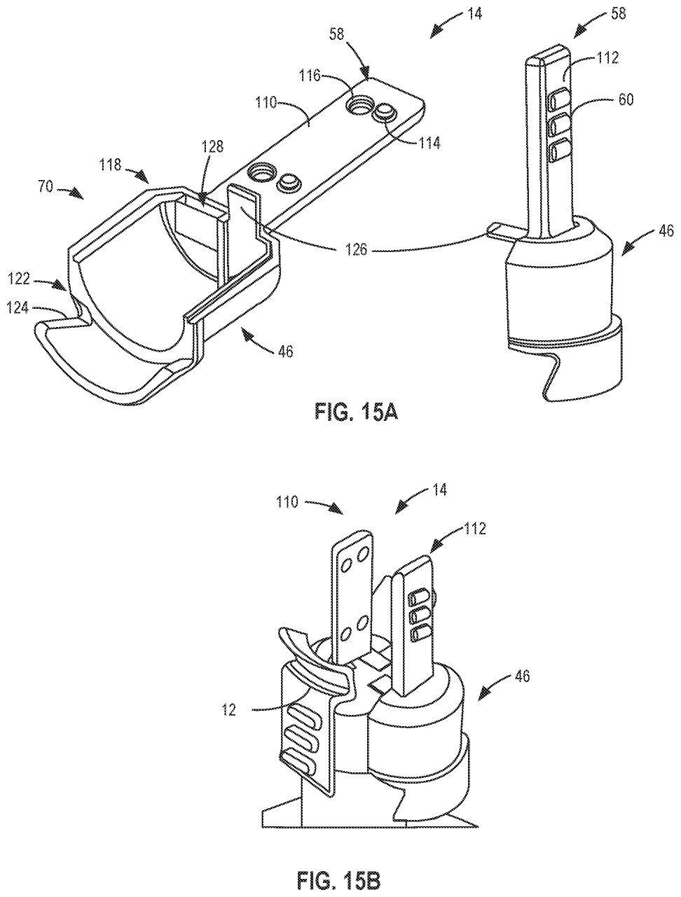

Referring to FIGS. 15A-15B, another embodiment of a two-piece dust cap 14 is illustrated. According to the illustrated embodiment, the two-piece dust cap 14 operates in a similar manner to the dust cap 14 shown in FIGS. 14A-14E, but may be assembled on the connector element 12 in a different manner. With reference to FIG. 15A, the first and second portions 110, 112 of the two-piece dust cap 14 may include the one or more alignment bosses 114, one or more alignment detent 116, and the at least one tongue 118 and the at least one groove 120 features as described herein and/or with reference to the various embodiment illustrated in FIGS. 14A-14E. Further, each of the first and second portions 110, 112 may include one or both of one or more male snap feature 126 and one or more female snap feature 128, where the one or more male and female snap features 126, 128 on one of the first portion 110 and the second portion 112 are paired with one or more complementary female and male snap features 128, 126 on the other of the first portion 110 and the second portion 112. In this manner, the first portion 110 and the second portion 112 of the dust cap 14 interface during engagement and may be held together by engagement of the one or more male snap feature 126 on the first portion 110 of the dust cap 14 and one or more corresponding female snap feature 128 on the second portion 112 of the dust cap 14. The first and second portions 110, 112 may be identical such that they can be manufactured using a single mold or may be different, e.g., as described herein and/or the first portion 110 may include one or more male snap feature 126 and the second portion 112 may include a corresponding one or more female snap feature 128 located and configured to engage the one or more male snap features 126 on first portion 110.

During assembly, the first and second portions 110, 112 of the two-piece dust cap 114 may be engaged with connector element 12 by laterally sliding each of the first and second portions 110, 112 between the opposing flexible legs 20, 22 of the connector element 12 as illustrated in FIG. 15B. As the first and second portions 110, 112 laterally approach, the one or more male snap feature 126 on one of the first portion 110 and the second portion 112 snaps into the one or more corresponding female snap feature 128 on the other of the first portion 110 and the second portion 112 to releasably engage the first and second portions 110, 112 of the two-piece dust cap 14. Removal of the dust cap similarly involves distally pulling on the grasping tab 58, which disengages the first portion 110 from the second portion 112 via the interaction of the tapered or angled lateral surface 124 on the undercut 122 of the first portion and second portion 110, 112 with the connector element 12 as described herein with reference to FIGS. 14A-14E. Tampering with, potential contamination of, or previous use of the syringe 16 may be indicated by observations of at least partial disconnection of first and second portions 110, 112 of the two-piece dust cap 14 or absence of dust cap 14 from connector element 12.

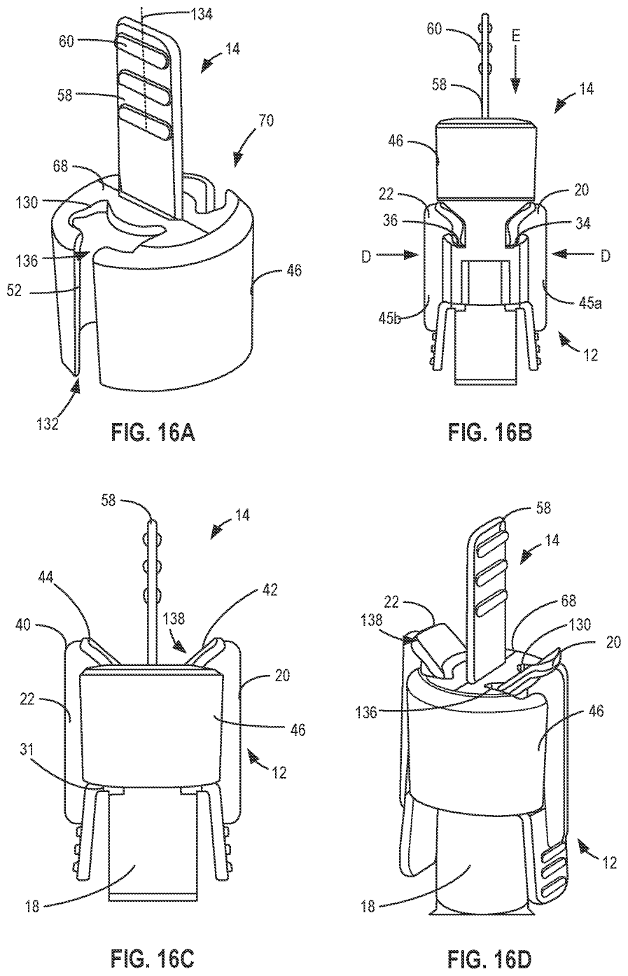

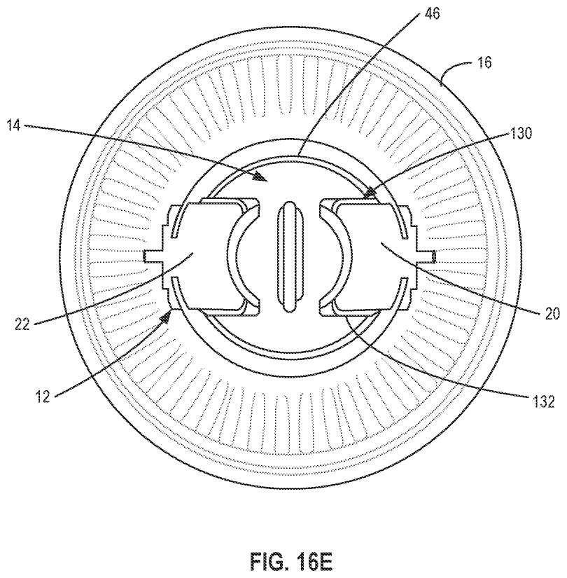

Referring to FIGS. 16A-16E, another embodiment of the dust cap 14 having a tamper resistant and/or tamper evident feature 70 is illustrated. The tamper resistant and/or tamper evident feature 70 may include at least one slot 130 extending through the sidewall 52. and ledge 68 of the dust cap body 46. The at least one slot 130 includes a first portion 132 extending through the sidewall 52 of the dust cap body 46 in a direction along a longitudinal axis 134 of the body 46 and a second portion 136 extending through the ledge 68 in a direction substantially perpendicular to the longitudinal axis 134. The at least one slot 130 may be a pair of slots 130 positioned opposite each other on opposites of the grasping tab 58.

The slots 130 may be configured to releasably engage distal surfaces 42, 44 of the flexible legs 20, 22 of the connector element 12. The second portion 136 of each slot 130 is configured to receive at least a portion of the distal surfaces 42, 44 of the first and second legs 20, 22 therethrough when the dust cap 14 is installed on the connector element 12. The first portion of 132 of each slot 130 is configured to receive the reinforcing member 45a, 45b of the first and second legs 20, 22, respectively. As shown in FIG. 16B, during installation of the dust cap 14 on the connector element 12, the flexible legs 20, 22 are pressed in a radially inward direction in a direction of arrows D such that the flange portions 34, 36 of the flexible legs 20, 22 can be received within the second portion 136 of the slots 130. As the dust cap 14 is moved in a proximal direction of arrow E in FIG. 16B, the reinforcing member 45a, 45b of each of the flexible legs 20, 22 slides within the first portion 132 of the corresponding slot 130. Once the dust cap 14 is pushed proximally toward the connector element 12 such that the dust cap body 46 engages the connection beam 31 of the connector element 12, distal surfaces 42, 44 of the flange portions 38, 40 of the flexible legs 20, 22 protrude from the second portion 136 of the slots 130, thereby providing clearance space for the flexible legs 20, 22 to elastically return in a radially outward direction to their original position. The dust cap 14 may be held on the distal end 21 of the syringe 16 by the radially outward engagement between the flange portions 38, 40 of the flexible legs 20, 22 and the inner surface of the sidewall 52 (shown in FIG. 16A) of the dust cap body 46.

In certain embodiments, the distal surfaces 42, 44 of the flexible legs 20, 22 may each include an angled trapping surface 138 on the end surfaces of flanges 34, 36. The trapping surface 138 is configured to engage and trap the ledge 68 of the dust cap body 46 onto the connector element 12. During removal of the dust cap 14 from the connector element 12, such as by distally pulling the grasping tab 58 of the dust cap 14 in a direction of arrow F in FIG. 16D, the portion of the sidewall 52 defining the second portion 136 of the slot 130 rides along the proximal surface of the angled trapping surface 138, thereby forcing the flexible legs 20, 22 to be moved in the radially inward direction and allowing the dust cap 14 to be slidably removed from the connector element 12. Reinstallation of the dust cap 14 onto connector element 18 is prevented due the required significant manipulation of the flexible legs 20, 22 and the dust cap 14. In this manner, tampering with, potential contamination of, or previous use of the syringe 16 may be indicated by absence of or dislocation of the dust cap 14 from the connector element 18.

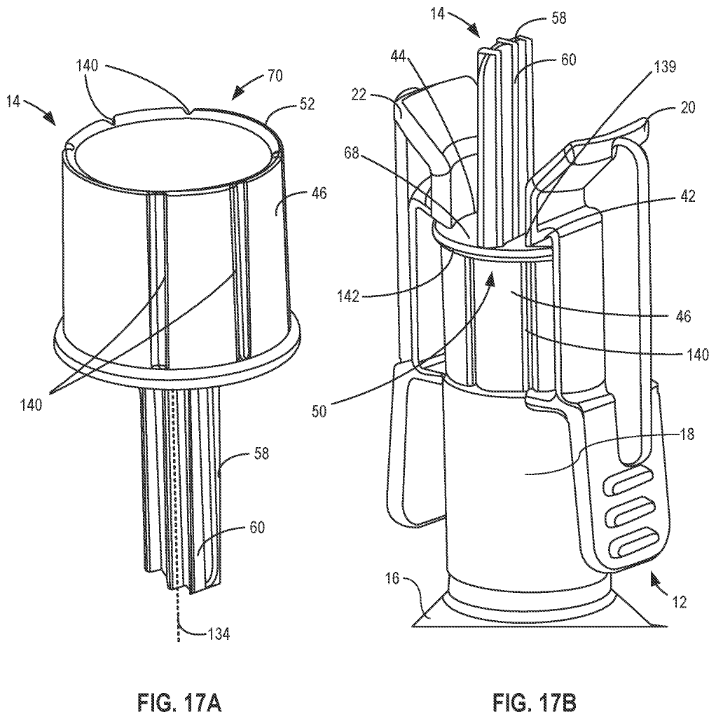

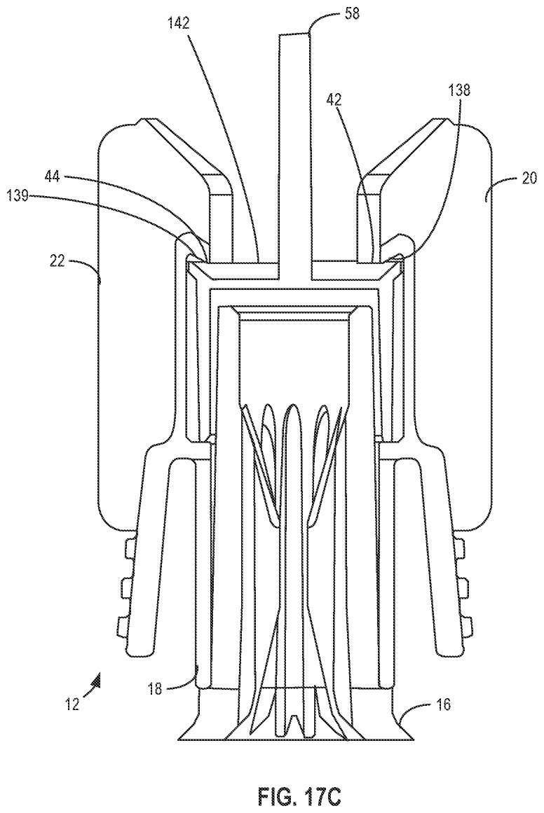

Referring to FIGS. 17A-17C, another embodiment of the dust cap 14 having a tamper resistant and/or tamper evident feature 70 is illustrated. According to this embodiment, the tamper resistant and/or tamper evident feature 70 may include one or more tearable seams 140 formed in the dust cap body 46. The one or more seams 140 may extend into an outer surface of the sidewall 52 of the dust cap body 46 to define a weakened portion that is breakable or tearable to allow removal of the dust cap 14 from the connector element 12. In some examples, the one or more seams 140 may extend in a direction substantially parallel with a direction of the longitudinal axis 134 of the dust cap 14. In other examples, the one or more seams 140 may be angled relative to the longitudinal axis 134 at an oblique angle. The seams 140 may be spaced apart from each other in equal or unequal angular intervals about an outer perimeter of the dust cap body 46.

With reference to FIGS. 17B-17C, the distal end 50 of the dust cap body 46 may have a flange 142 that protrudes distally relative to the ledge 68. The flange 142 may be angled radially outward relative to the sidewall 52 of the dust cap body 14. The distal surfaces 42, 44 of the flexible legs 20, 22 may each include the angled trapping surface 139 on the end surfaces of flanges 34, 36 that are configured to capture the flange 142 when the dust cap 14 is connected to the connector element 12. In other embodiments, distal end 50 of the dust cap 14 may include a sloping member 64 defined by a distal edge 66 of dust cap body 46, as described herein.

When a twisting or sideways force is applied to the dust cap 14, such as twisting the grasping tab 58 about the longitudinal axis 134 or moving the grasping tab 58 in a direction perpendicular or oblique to the longitudinal axis 134, the one or more seams 140 is configured to separate, split, and/or tear along the sidewall 52 of the dust cap body 46, so that the dust cap 14 may be readily removed from the connector element 12, for example by laterally sliding the dust cap 14 to the side of the connector element 12 or distally pulling the grasping tab 58 in a direction substantially along the longitudinal axis. As the material of the dust cap 14 is now torn, the dust cap 14 cannot be reinstalled onto the connector element 12. In this manner, tampering with, potential contamination of, or previous use of the syringe 16 may be indicated by at least one torn seam 140.

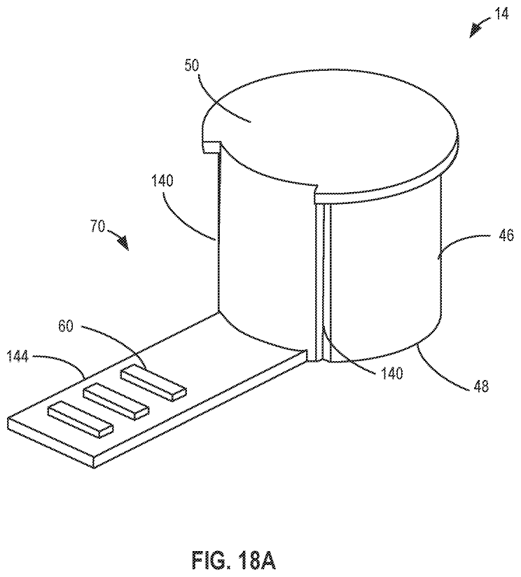

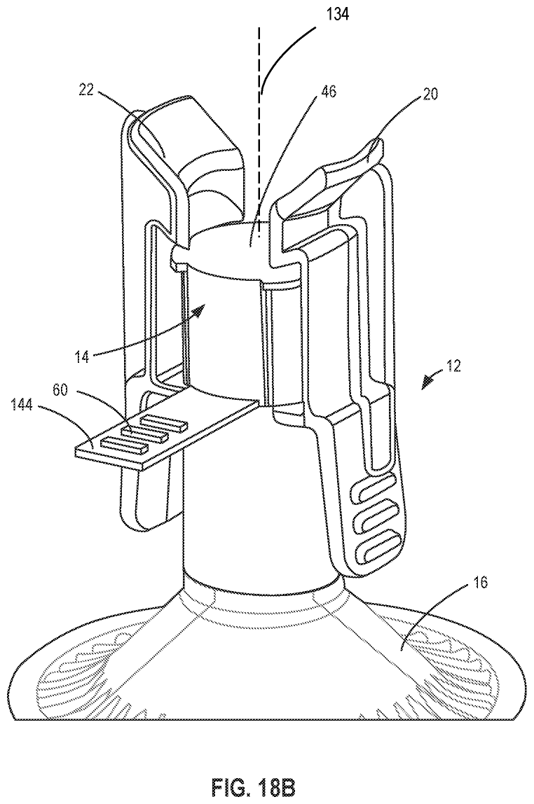

According to another embodiment, as shown in FIGS. 18A-18B, a tamper resistant and/or tamper evident feature 70 may include a dust cap 14 having two or more tearable seams 140 molded or cut into the sidewall of the dust cap body 46 in a manner similar as described with reference to FIGS. 17A-17C. The two or more seams 140 extend from the proximal end 48 to the distal end 50 of the dust cap body 46 in a direction substantially parallel with a direction of the longitudinal axis 134. In some examples, the two or more seams 140 may be angled relative to the longitudinal axis 134. The embodiment shown in FIGS. 18A-18B further has a pull tab 144 extending away from the dust cap body 46 between the two or more seams 140. Pulling on the pull tab 144 is configured to separate or tear the seams 140. In this manner, the dust cap 14 can be removed from the connector element 12 by pulling the dust cap 14 sideways between the flexible legs 20, 22 or pulling the pull tab 144 over the top of the dust cap 14. As the material of the dust cap 14 is now torn, this provides indication that the dust cap 14 has been previously removed and should not be reinstalled onto the connector element 12. In this manner, tampering with, potential contamination of, or previous use of the syringe 16 may be indicated by the torn seams 140.

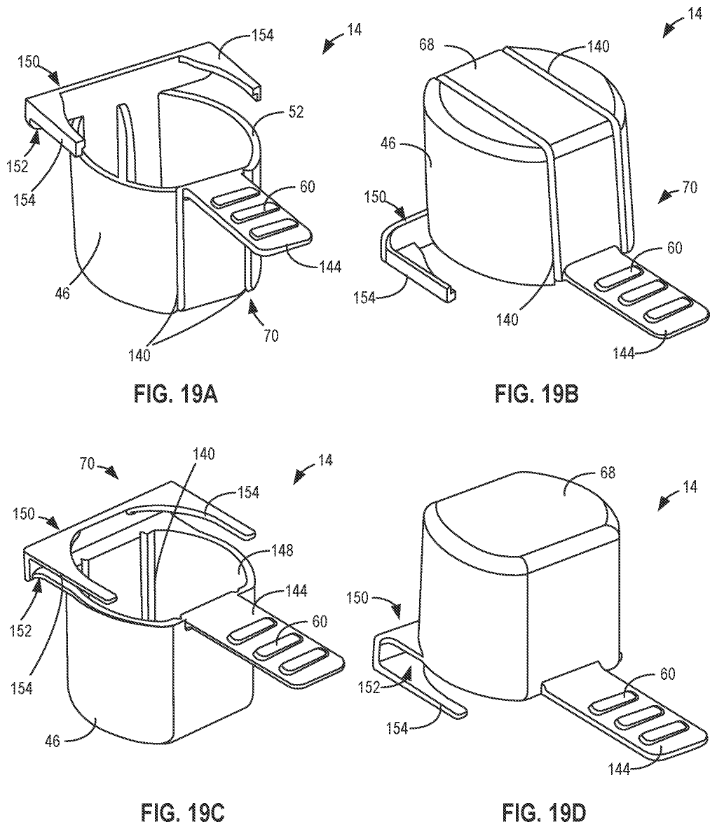

According to other embodiments, as shown in FIGS. 19A-19D, a tamper resistant and/or tamper evident feature 70 may include a dust cap 14 having two or more seams 140 molded or cut into an exterior (FIGS. 19A, 19B) or an interior (FIGS. 19C, 19D) of the sidewall 52 of the dust cap body 46 in a manner similar as described with reference to FIGS. 18A-18B. The two or more seams 140 may extend from one side of the body 46 to an opposing side of the body 46 or may extend from one side of the body 46 over ledge 68 of dust cap 14. In some examples, such as shown in FIGS. 19A-19B, the two or more seams 140 extend partially into the sidewall 52 on an outer surface 146 of the body 46. In other examples, such as shown in FIGS. 19C-19D, the two or more seams 140 extend partially into the sidewall 52 on an inner surface 148 of the body 46. A pull tab 144 may extend away from the dust cap body 46 between the two or more seams 140 such that pulling on the pull tab 144 is configured to separate or tear the seams 140 and allow removal of dust cap 14.

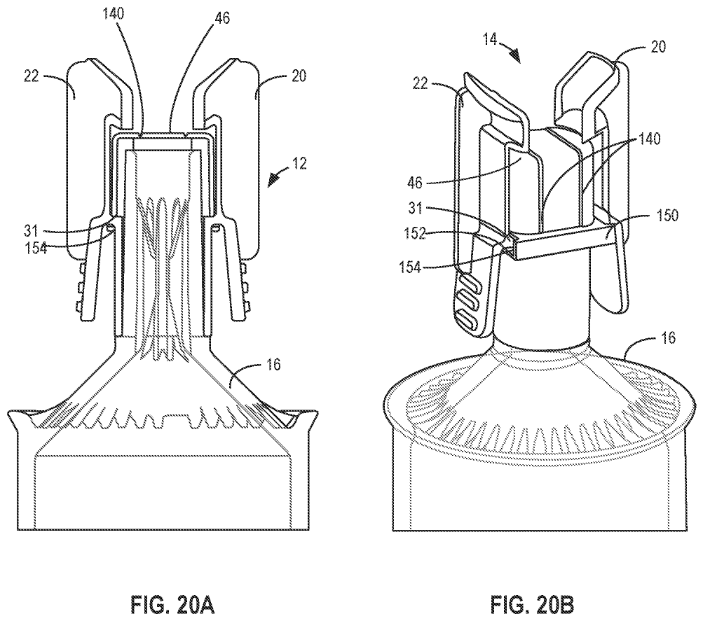

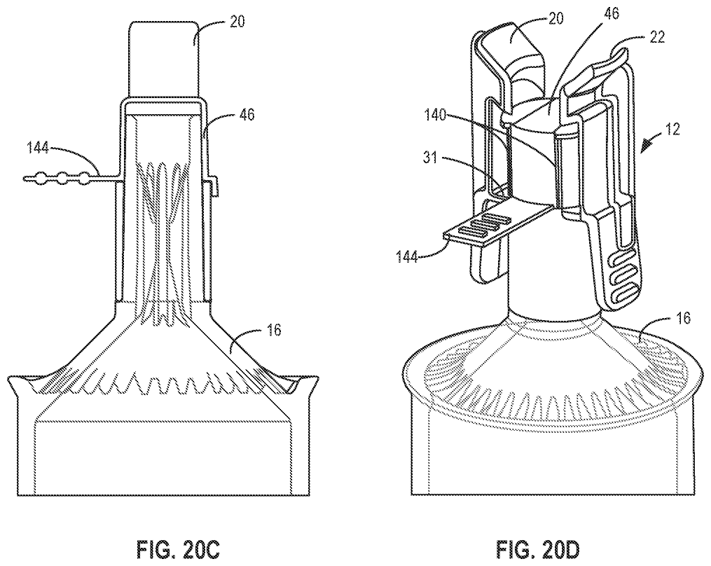

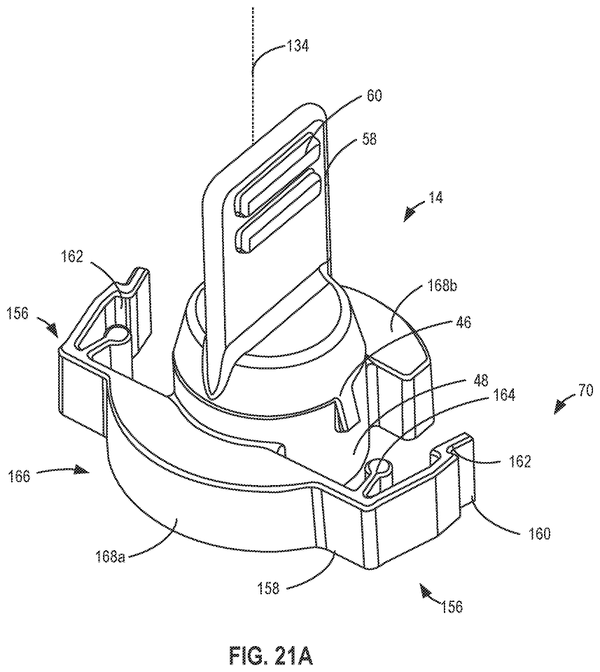

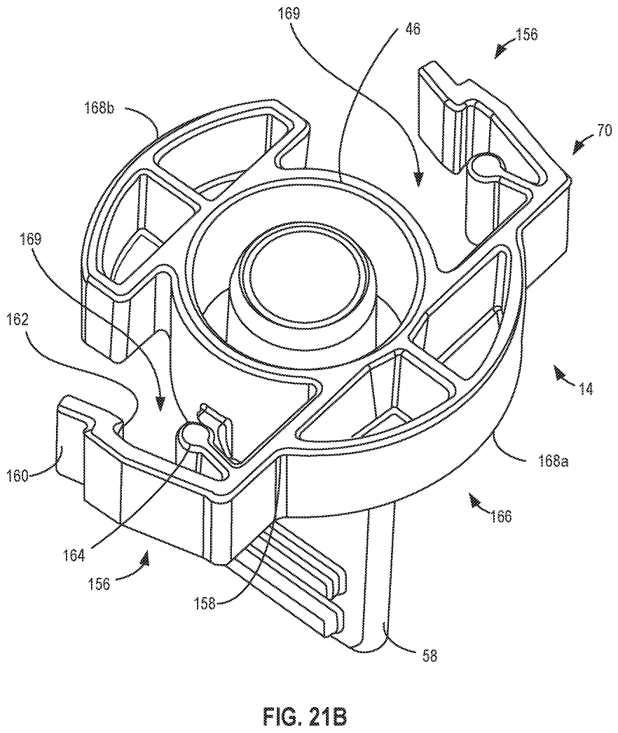

With continued reference to FIGS. 19A-19D, the tamper resistant and/or tamper evident feature 70 may include a retention flange 150 extending proximally from the proximal end 48 of the dust cap body 46. The retention flange 150 has a retention pocket 152 defined by a pair of retention legs 154 that are spaced apart from the proximal end 48 of the dust cap body 46. In some examples, and with reference to FIGS. 20A-20D, the retention pocket 152 is configured to receive at least a portion of the connection beam 31 of the flexible legs 20, 22 when the dust cap 14 is connected to the connector element 12. The retention flange 150 is configured to prevent the dust cap 14 from distally sliding off the connector element 12 during manufacturing, shipping, or storage and further may require pulling on pull tab 144 and tearing of seams 140 to remove the dust cap 14.

Pulling on the pull tab 144 is configured to separate or tear the seams 140. In this manner, the dust cap 14 can be removed from the connector element 12 by pulling the dust cap 14 sideways or upwards between the flexible legs 20, 22 such that the retention feature 150 slides relative to the connection beam 31. As the material of the dust cap 14 is now torn, this provides indication that the dust cap 14 should not be reinstalled onto the connector element 12. In this manner, tampering with, potential contamination of, or previous use of the syringe 16 may be indicated by the torn seams 140.

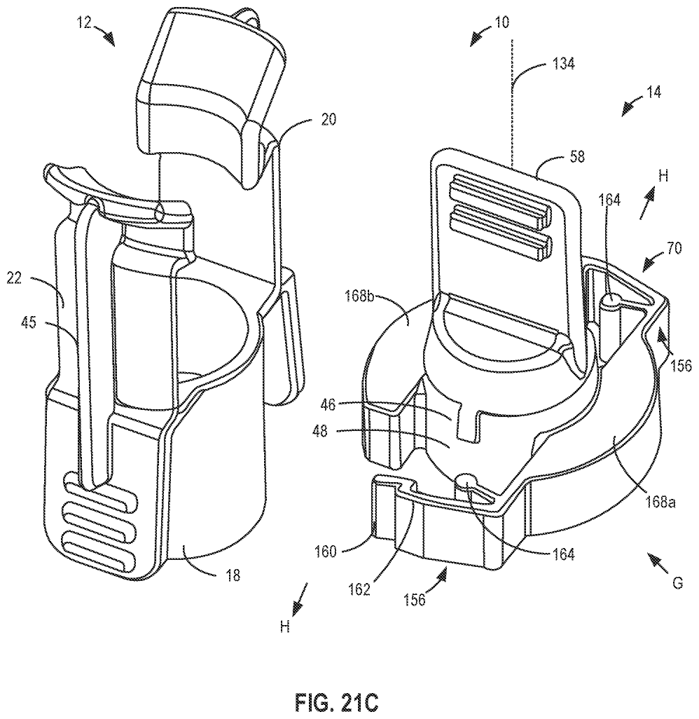

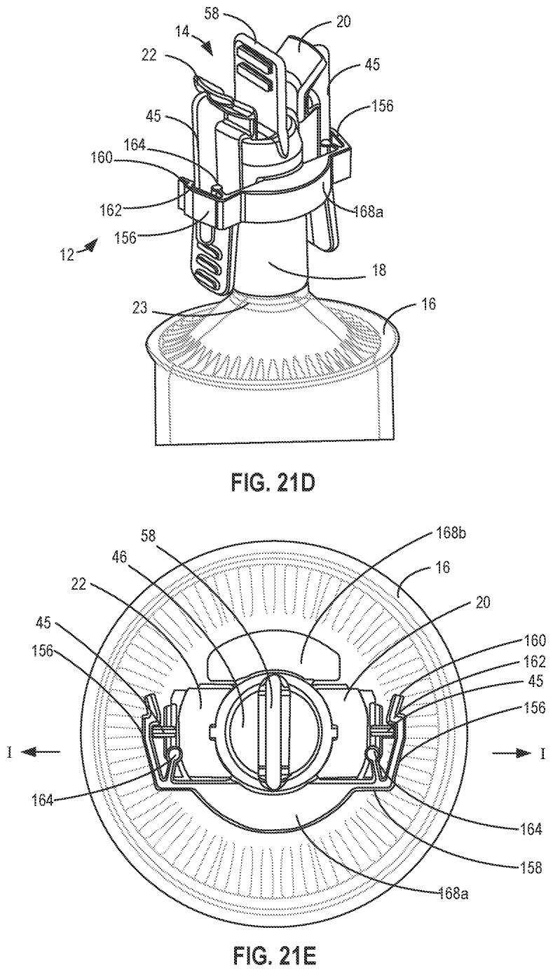

According to another embodiment, as shown in FIGS. 21A-21E, a tamper resistant and/or tamper evident feature 70 may include a dust cap 14 that may be installed onto the connector element 12 prior to the connector element 12 being attached to the syringe 16. As illustrated in FIGS. 21A and 21B, the dust cap 14 may have one or more side arms 156 connected to the dust cap body 46 and configured to engage the flexible first and second legs 20, 22 of the connector element 12. Each of the one or more side arms 156 has a first end 158 connected to the dust cap body 46 and a second end 160 protruding in a direction substantially perpendicular to the longitudinal axis 134. In some examples, the first end 158 of the one or more side arms 156 may be integrally formed with the dust cap body 46 at the proximal end 48 of the dust cap body 46. The one or more side arms 156 may be made of a material such that they can pivot or flex at the first end 158 to allow the second end 160 to move radially outward in a direction relative to a longitudinal axis 134. In one example of the present disclosure, the one or more side arms 156 may be made of a rigid material with a flexible pivot point to allow the one or more side arms 156 to move radially outward in a direction relative to a longitudinal axis 134.

The dust cap 14 is installed onto the connector element 12 by sliding the dust cap 14 laterally onto the connector element 12 in a direction of arrow G in FIG. 21C such that the second end 160 of the one or more side arms 156 can flex around and engage with the first and second flexible legs 20, 22 of the connector element 12 for example by flexing over reinforcing member 45. For example, as the dust cap 14 is moved toward the connector element 12 in the direction of arrow G, the second end 160 of the side arms 156 is configured to flex radially outward in a direction of arrows H to allow the side arms 156 to extend around the reinforcing member 45 of the first and second flexible legs 20, 22 of the connector element 12. The second end 160 of the side arms 156 is configured to revert to its unflexed position after clearing the reinforcing member 45 of the first and second flexible legs 20, 22 such that the reinforcing member 45 is retained within a retaining notch 162 on the second end 160 of the one or more side arms 156 (see FIGS. 21D-21E).

The side arms 156 may each have a tab 164 connected to the side arms 156 between the first end 158 and the second end 158 (see FIG. 21B). In some examples, the tab 164 may extend in a direction substantially parallel with the side arm 156. The tab 164 may be configured to engage the side of the first or second leg 20, 22 and abut the reinforcing member 45. During removal of the dust cap 14 from the connector element 12, the tab 164 may be configured to flex over the distal end of the first and second flexible legs 20, 22 in a direction of arrows I in FIG. 21E. After removal of the dust cap 14 from the connector element 12, the tab 164 is configured to revert to its original, unflexed position. The tab 164 is configured to prevent reinstallation of the dust cap 14 onto the connector element 12 after removal, for example by preventing the user from proximally pushing the dust cap 14 back onto the connector element 12. In some examples, the tab 164 may be positioned such that the tab 164 interferes with the outwardly angled distal ends of the first and second legs 20, 22.

The dust cap 14 further may include a skirt 166 extending around at least a portion of the dust cap body 46. The skirt 166 may extend in a radially outward direction relative to the dust cap body 46. In some examples, the skirt 166 may extend in a radially outward direction from the proximal end 48 of the dust cap body 46. In some examples, the skirt 166 may have a first portion 168a separated from a second portion 168b such that the first and second portions 168a, 168b extend around a portion of an outer perimeter of the dust cap body 46. In some examples, the first end 158 of the one or more side arms 156 may be connected to the first portion 168a. The first and second portions 168a, 168b of the skirt 166 may be separated by one or more gaps 169 (shown in FIG. 21B) that is configured to accommodate the first and second flexible legs 20, 22 when dust cap 14 is installed on connector element 12.

Once the dust cap 14 is installed onto the connector element 12 as described herein, the connector assembly 10 may then be attached, for example by laser welding or adhering with a glue, to the syringe 16 such that the dust cap 14 seals the discharge nozzle 23 of the syringe 16, as shown in FIGS. 21D and 21E. To remove the dust cap 14 from the connector element 12, the user removes the dust cap by pulling the grasping tab 56 of the dust cap 14 distally along the longitudinal axis 134 of the connector element 12, thereby urging the side arms 156 and tab 164 to flex and slide over the outer surfaces and the reinforcing members 45 of the first and second legs 20, 22 of the connector element 12. As described, the tab 164 also flexes and passes the distal end of the first and second flexible legs 20, 22 during the removal process, and is then configured to flexibly return or snap back to its original orientation to prevent reinstallation of the dust cap 14 onto the connector element 12.

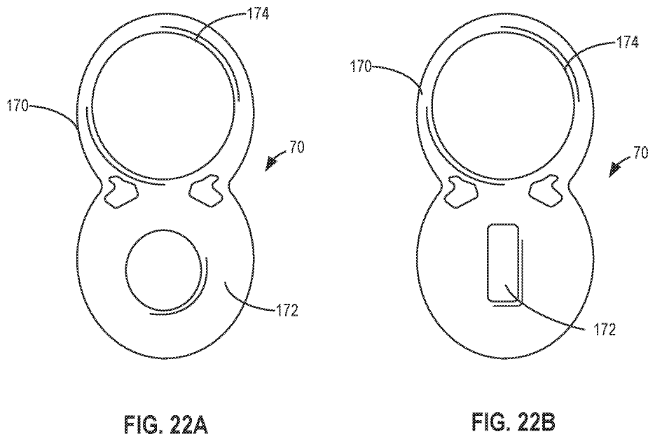

According to another embodiment, as shown in FIGS. 22A-22B, a tamper resistant and/or tamper evident feature 70 may include a flexible strip 170 having a first opening 172 configured to receive at least a portion of the dust cap 14 and a second opening 174 configured to receive at least a portion of the connector element 12. In some examples, such as shown in FIG. 22A, the first and second openings 172, 174 may have a circular shape. In other examples, at least one of the first and second openings 172, 174 may have a non-circular shape. In some examples, the first opening 172 may be shaped to correspond to a shape of at least a portion of the dust cap 14, such as a circular portion of the ledge 68 (FIG. 22A) or grasping tab 58 (see FIG. 22B). Similarly, the second opening 174 may be shaped to correspond to at least a portion of the connector element 12, such as the body 18 of the connector element 12. The flexible strip 170 is configured to bend or flex such that at least a portion of the dust cap 14 is received in the first opening 172 and such that at least a portion of the connector element 12 is received in the second opening 174. The flexible strip 170 is configured to prevent removal of the dust cap 14 from the connector element 12 without first removing the flexible strip 170.

In some examples, the flexible strip 170 may be made from a tearable plastic, paper, or other readily tearable textile material. To remove the flexible strip 170 from the dust cap 14 and the connector element 12, the user can simply pull on the flexible strip 170, thereby causing at least a portion of the flexible strip 170 to break or tear. For example, portions of the flexible strip 170 surrounding the first and second openings 172, 174 may be configured to tear when the flexible strip 170 is pulled in a sideways direction, such as in a radial direction relative to the dust cap 14 and the connector element 12. Alternatively, removal of the dust cap 14, for example by grasping and pulling the grasping tab 58 in a longitudinal distal direction, may remove the dust cap 14 while stretching and tearing flexible strip 170. Once the material of the flexible strip 170 is torn, the dust cap 14 can be removed from the connector element 12. Tampering with, potential contamination of, or previous use of the syringe 16 may be indicated by the torn flexible strip 170.

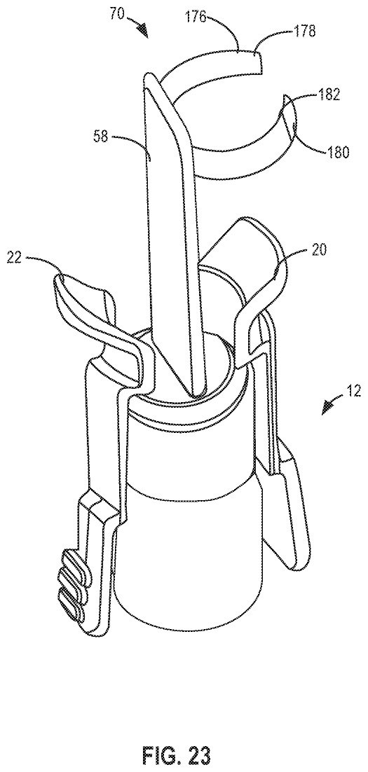

With reference to FIG. 23, and according to another example, a tamper resistant and/or tamper evident feature 70 may include a strap 176 connected to the dust cap 14, such as the grasping tab 58 of the dust cap 14. In some examples, the strap 176 may be integrally formed with the grasping tab 58 of the dust cap 14, such as by being molded therewith. In other embodiments, the 176 may be adhered to or welded to the grasping tab 58 of the dust cap 14. The grasping tab 58 may be flexible or bendable such that it can be bent over to be positioned proximate to connector element 12 to allow strap 176 to be connected to connector element 12.

In some examples, the strap 176 may be a zip tie having a first free end 178 connectable with a connector 180 on a second free end 182. The zip tie may be configured to wrap around a perimeter of the connector element 12 such that the first free end 178 can be inserted through the connector 180 of the second free end 182 to secure the zip tie to the connector element 12. In this way, the dust cap 14 can be securely connected with the connector element 12, with the strap 176 preventing removal of the dust cap 14 without disconnection of the tamper resistant and/or tamper evident feature 70, for example by breaking the strap 176 from the connector element 12 and/or the dust cap 14. According to another embodiment, strap 176 may be substantially circular and may be configured to surround the body 18 of the connector element 12 prior to connection of the connector element 12 to the syringe 16. Once the connector element 12 is engaged with the syringe 16, the strap 176 cannot be removed from the body 18 of the connector element 12 without tearing the strap and the dust cap 14 cannot be removed unless the strap 176 is broken or the grasping tab 58 is broken. The strap 176 may be broken by pulling or rotating the strap 176 to break the connection between the strap 172 and the dust cap 14 and/or the connector element 12. In some embodiments, removal of the dust cap 14 in a distal direction may break strap 176. Tampering with, potential contamination of, or previous use of syringe 16 may be indicated by the absence of or damage to strap 176.

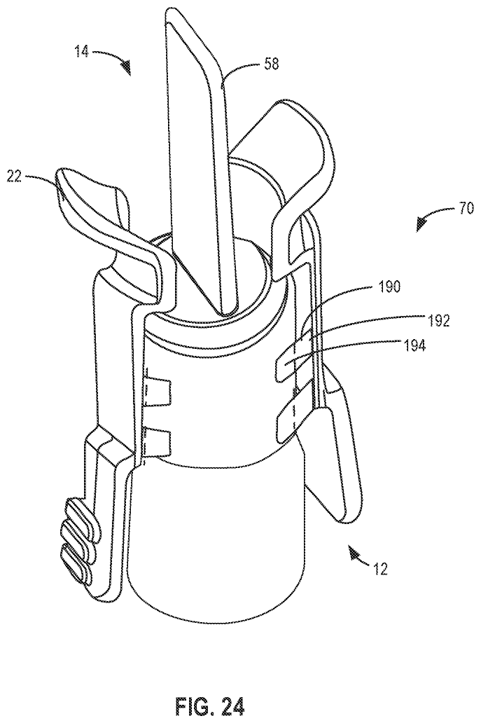

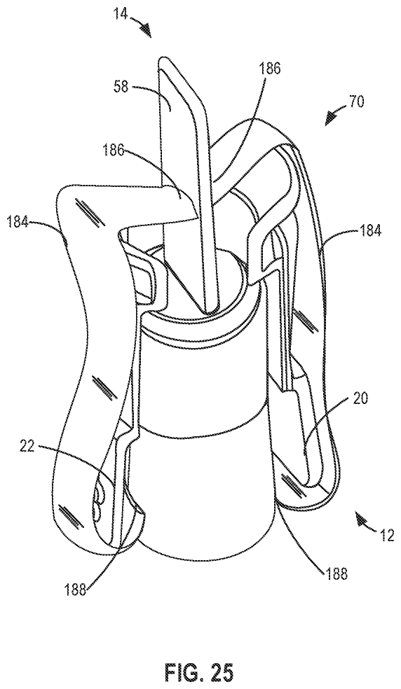

With reference to FIG. 24, and according to another example, a tamper resistant and/or tamper evident feature 70 may include at least one breakable beam 190 connecting the connector element 12, such as one of first and second flexible legs 20, 22, to the dust cap 14. In some examples, the at least one breakable beam 190 has a first end 192 connected to one of the first flexible leg 20 and the second flexible leg 22 and a second end 194 connected to at least a portion of the dust cap 14, such as the dust cap body 46 or the grasping tab 58. When the breakable beam 190 is connected to the connector element 12 and the dust cap 14, the breakable beam 190 prevents movement of the dust cap 14 relative to the connector element 12 in a distal direction. By rotating the dust cap 14 about the longitudinal axis 134, at least one of the first and second ends 192, 194 is configured to break away from the first/second flexible leg 20, 22 and/or the dust cap 14, thereby permitting removal of the dust cap 14 via pulling in a distal direction. Tampering with, potential contamination of, or previous use of the syringe 16 may be indicated by a broken state of the breakable beam 190.

With reference to FIG. 25, and according to another example, a tamper resistant and/or tamper evident feature 70 may include at least one strip 184, such as a pair of strips 184, each having a first end 186 connected to at least a portion of the dust cap 14 and a second end 188 connected to a portion of the connector element 12. In some examples the first end 186 may be connected to the grasping tab 58 of the dust cap 14, while the second end 188 may be connected to the proximal gripping portion of the flexible first and second legs 20, 22. In other embodiments, the second end 188 may be attached to a flange or ledge (not shown) integrally formed on an outer surface of the flexible first and second legs 20, 22. In some examples, each of the strips 184 may be integrally formed with the grasping tab 58 of the dust cap 14 or the connector element 12, such as by being molded therewith. The dust cap 14 can be securely connected with the connector element 12, with the strips 184 preventing removal of the dust cap 14 without disconnection of the tamper resistant and/or tamper evident feature 70, for example by breaking the strips 184 from the connector element 12 and/or the dust cap 14. The strip 184 may be broken by pulling or rotating the strips 184 or pulling or rotating the dust cap 14 to break the connection between the strips 184 and the dust cap 14 and/or the connector element 12. Tampering with, potential contamination of, or previous use of the syringe 16 may be indicated by the absence of or damage to the strips 184.

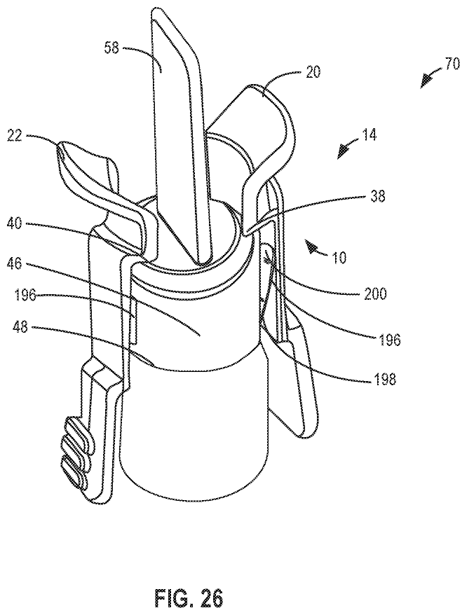

With reference to FIG. 26, and according to another example, a tamper resistant and/or tamper evident feature 70 may include a skirt 196 connected to the proximal end 48 of the dust cap 14. In some examples, the skirt 196 may be an annular skirt. In other examples, the skirt 196 may extend at least partially around the dust cap body 46. The skirt 196 may have a first end 198 connected to the proximal end 48 of the dust cap body 46 and a second free end 200. In some examples, the second free end 200 is folded over such that the skirt 196 extends in a distal direction when the dust cap 14 is installed on the connector element 12. In some examples, the second end 200 of the skirt 196 may be configured to be received within the first and second flanges 38, 40 of the first and second legs 20, 22 of the connector element 12. When the dust cap 14 is connected to the connector element 12, the arrangement of the second end 200 of the skirt 196 within the space of the first and second flanges 38, 40 prevents movement of the dust cap 14 relative to the connector element 12 in a distal direction without deformation of the second end 200 relative to the first end 198. Pulling the dust cap 14 in a distal direction, such as by pulling the grasping tab 58, the skirt 196 is configured to unfold, for example by deforming and unfolding as the second end 200 abuts the first and second flanges 38, 40 of the first and second legs 20, 22 with the second end 200 being forced to move in the proximal direction relative to the first end 198 as the dust cap 14 continues to be moved in distal direction, thereby permitting removal of the dust cap 14 via pulling in the distal direction. Tampering with, potential contamination of, or previous use of the syringe 16 may be indicated by an unfolded state of the skirt 196 on the dust cap 14.

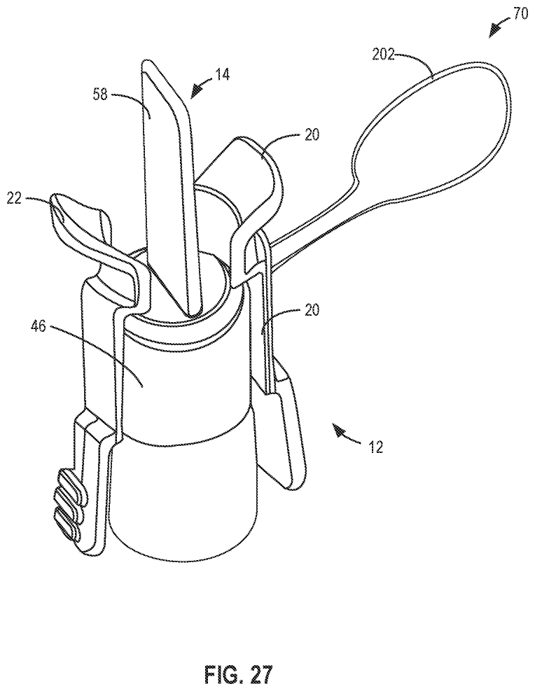

With reference to FIG. 27, and according to another example, a tamper resistant and/or tamper evident feature 70 may include at least one loop 202 that is connected to one of the first flexible leg 20 and the second flexible leg 22 of the connector element 12. In some examples, the at least one loop 202 may be integrally formed with one of the first flexible leg 20 and the second flexible leg 22, such as by being molded therewith, or alternatively the at least one loop 202 may be adhesively connected or welded to one of the first flexible leg 20 and the second flexible leg 22. In some examples, the at least one loop 202 may be elastic, such that it may be stretched from the first one of the first flexible leg 20 and the second flexible leg 22 and looped over the other of the first flexible leg 20 and the second flexible leg 22 in order to surround an outer perimeter of at least a portion of the first and second flexible legs 20, 22. The presence of the at least one loop 202 around the first and second flexible legs 20, 22 prevents movement thereof in the radially outward direction. In this manner, if removal of the dust cap 14 is attempted while the at least one loop 202 is connected, the at least one loop 202 prevents the radial deflection of the first and second flexible legs 20, 22 and thereby prevents the removal of the dust cap 14. When the at least one loop 202 is removed from looping over the first and second flexible legs 20, 22, such as by breaking the at least one loop 202 for example by medical personnel pulling the material of the at least one loop 202 radially outward, the flexible legs 20, 22 may move in a radially outward direction as the dust cap 14 is removed from the connector element 12, as described herein. In further examples, removal of the dust cap 14 while the at least one loop 202 is engaged with the first and second flexible legs 20, 22 may break the at least one loop 202, thereby providing an indication that the connector element 12 may have been used. Tampering with, potential contamination of, or previous use of the syringe 16 may be indicated by the absence of or damage to the at least one loop 202.

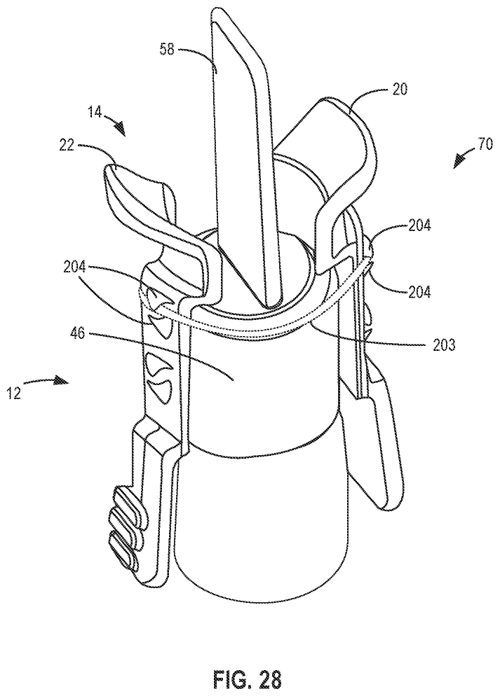

With reference to FIG. 28, on or both of the first and second flexible legs 20, 22 may have one or more notches 204 configured to retain a band 203 that extends around a perimeter of the first and second flexible legs 20, 22. The one or more notches 204 may be configured as protrusions that extend radially outward from an outer surface of the first and second flexible legs 20, 22 to receive the band 203. In some examples, a plurality of notches 204 may be spaced apart axially along the longitudinal length of the first and second flexible legs 20, 22. The band 203 may be elastic, such that it may be stretched from the first one of the first flexible leg 20 and the second flexible leg 22 and looped over the other of the first flexible leg 20 and the second flexible leg 22 and secured within the one or more notches 204 in order to surround an outer perimeter of at least a portion of the first and second flexible legs 20, 22. In some examples, the band 203 may be positionable in any of the notches 204 along the longitudinal length of the first and second flexible legs 20, 22. The presence of the band 203 around the first and second flexible legs 20, 22 prevents movement thereof in the radially outward direction. In this manner, if removal of the dust cap 14 is attempted while the band 203 is connected, the band 203 prevents the radial deflection of the first and second flexible legs 20, 22 and thereby prevents the removal of the dust cap 14. When the band 203 is removed from looping over the first and second flexible legs 20, 22, such as by breaking the band 203 for example by medical personnel pulling the material of the band 203 radially outward, the flexible legs 20, 22 may move in a radially outward direction as the dust cap 14 is removed from the connector element 12, as described herein. In further examples, removal of the dust cap 14 while the band 203 is engaged with the first and second flexible legs 20, 22 may break the band 203, thereby providing an indication that the connector element 12 may have been used. Tampering with, potential contamination of, or previous use of the syringe 16 may be indicated by the absence of or damage to the band 203.

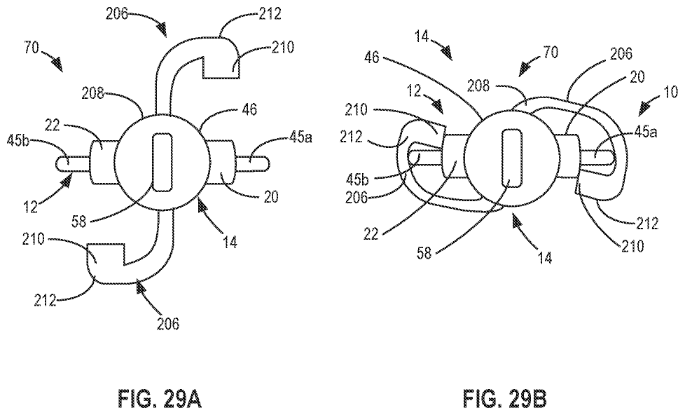

With reference to FIGS. 29A-29B, and according to another example, a tamper resistant and/or tamper evident feature 70 may include at least one flexible locking leg 206 connected to the dust cap 14, such as the body 46 of the dust cap 14. In some examples, the at least one flexible locking leg 206 may be a pair of flexible locking legs 206 extending radially outward from the dust cap body 46. Each flexible locking leg 206 has a first end 208 connected to the dust cap body 46 and a second end 210 protruding radially outward relative to the first end 208. In some examples, the second end 210 may have an engagement hook 212 configured for connecting with the connector element 12. For example, the engagement hook 212 may be configured to extend around at least a portion of the first flexible leg 20 or the second flexible leg 22, for example by hooking around a surface of the reinforcing members 45a, 45b on the first flexible leg 20 and the second flexible leg 22, respectively. The at least one flexible locking legs 206 may be movable between a disengaged position (FIG. 29A), in which the at least one flexible locking legs 206 are not connected to one or both of the first and second flexible legs 20, 22, and an engaged position (FIG. 29B), in which the at least one flexible locking legs 206 are in hooked engagement with one or both of the first and second flexible legs 20, 22. The locking legs 206 are movable between the disengaged and engaged positions via rotational movement of the dust cap 14 about the longitudinal axis. For example, rotation of dust cap 14 may deform the material associated with the engagement hook 212 so that the second end 210 of the at least one flexible locking leg 206 disengages the at least a portion of the first flexible leg 20 or the second flexible leg 22. Alternatively, the user may manually disengage the engagement hook 212 from the at least a portion of the first flexible leg 20 or the second flexible leg 22 prior to removal of the dust cap 14. In the engaged position, the locking legs 206 prevent removal of the dust cap 14 from the connector element 12. In the disengaged position of the locking legs 206, the dust cap 14 can be removed from the connector element 12. Tampering with, potential contamination of, or previous use of the syringe 16 may be indicated, for example, by deformation of the engagement hook 212 on the second end 210 of the at least one flexible locking leg 206 of the dust cap 14.

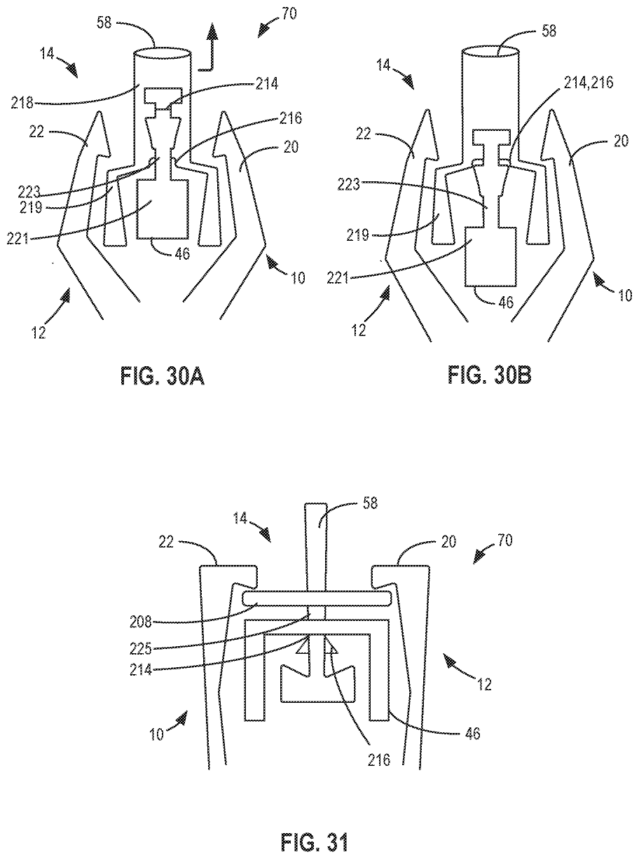

With reference to FIGS. 30A-30B, and according to another example, a tamper resistant and/or tamper evident feature 70 may include the dust cap 14 having a moveable element 218 movable relative to the dust cap body 46. The movable element 218 may include an outer cylindrical skirt 219 configured to surround an inner cylindrical portion 221 of the dust cap body 46 that is configured to fit within the nozzle of the connector element 12. In some examples, the grasping tab 58 may be defined on the movable element 218. The moveable element 218 may have a first engagement element 216. At least a portion of the dust cap body 46 is received within the interior of the outer cylindrical skirt 219. The distal portion of the dust cap body 46 has a second engagement element 214 that is configured to engage with the first engagement element 216 of the movable element 218 when the movable element 218 is moved to its distal-most position relative to the dust cap body 46.

The dust cap body 46 may include a post 223 extending through the first engagement element 216 such that, in an engaged position, the second engagement element 214 is locked with the first engagement element 216 (FIG. 30B). In this manner, the movable element 218 is freely movable relative to the dust cap body 46 in the direction of arrow F until the first engagement element 216 in in locking engagement with the second engagement element 214. Once the first and second engagement elements 216, 214 are lockingly engaged, continued movement of the movable element 218 relative to the dust cap body 46 in the distal direction results in removal of the dust cap body 46 from the connector element 12, such as by radial displacement of the flexible legs 20, 22, as described herein. When the first and second engagement elements 216, 214 are engaged, they are configured to remain engaged. In this manner, a locking engagement between the moveable element 218 and the dust cap body 46 is an indication that the dust cap 14 was removed.

In one embodiment, the second engagement element 214 may be in the form of angled fins extending from post 223 and the first engagement element 216 is in the form of a hole through which the post 223 extends. As the grasping tab 58 is pulled in the distal direction, the angled fins of the second engagement element 214 contact and lock with the outer circumference of the hole of first engagement element 216 to lock dust cap 14 in the extended position. Tampering with, potential contamination of, or previous use of syringe 16 may be indicated by a locking engagement between movable element 218 and dust cap body 46.

With reference to FIG. 31, and according to another example, a tamper resistant and/or tamper evident feature 70 may include the dust cap 14 having a moveable element 225 movable relative to the dust cap body 46, similar to the movable element 218 as described herein with reference to FIGS. 30A-30B. The movable element 225 is surrounded by the dust cap body 46 and is received within an opening in the dust cap body 46 such that the movable element 225 is movable relative to the dust cap body 46. In some examples, the grasping tab 58 may be defined on the movable element 225. The moveable element 225 may have a first engagement element 216, while the distal portion of the dust cap body 46 has a second engagement element 214 in the form of an opening on the dust cap body 46 that is configured to engage with the first engagement element 216 of the movable element 225 when the movable element 225 is moved to its distal-most position relative to the dust cap body 46, as described herein with reference to FIGS. 30A-30B. The tamper resistant and/or tamper evident feature 70 further includes a disc 208 connected to the grasping tab 58 to engage the dust cap body 46 with the flexible legs 20, 22 of connector element 12 holding the dust cap 14 on the connector element 12 during shipping and storage.

The movable element 225 is freely movable relative to the dust cap body 46 in the distal direction until the first engagement element 216 in in locking engagement with the second engagement element 214. Once the first and second engagement elements 216, 214 are lockingly engaged, continued movement of the movable element 225 relative to the dust cap body 46 in the distal direction results in removal of the dust cap body 46 from the connector element 12, such as by radial displacement of the flexible legs 20, 22, as described herein. When the first and second engagement elements 216, 214 are engaged, they are configured to remain engaged. In this manner, a locking engagement between the grasping tab 58 and the dust cap body 46 is an indication that the dust cap 14 was removed. The disc 208 may be configured to prevent reengagement of the dust cap 14 with the connector element 12 due to interference with the first and second flexible legs 20, 22 when the first and second engagement elements 216, 214 are engaged, locking the moveable element 225 in the distal most position and preventing the disc 208 from reengaging the first and second flexible legs 20, 22. Tampering with, potential contamination of, or previous use of the syringe 16 may be indicated by a locking engagement between the first engagement elements 216 on movable element 225 and second engagement element 214 on the dust cap body 46.

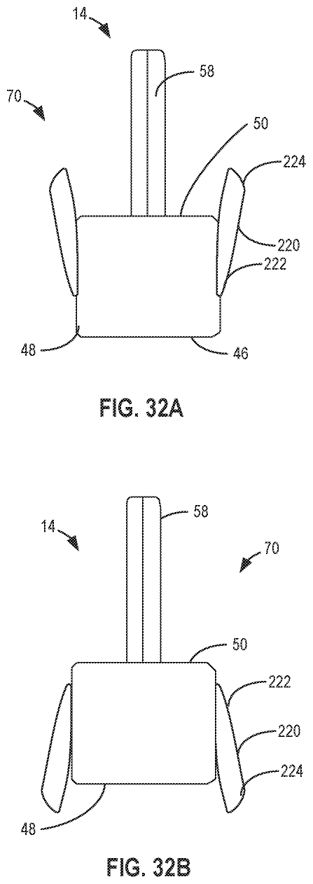

With reference to FIGS. 32A-32B, and according to another example, a tamper resistant and/or tamper evident feature 70 may include a deformable flange 220 connected to the dust cap body 46 between the proximal end 48 and the distal end 50. In some examples, the deformable flange 220 may be annularly shaped. In other examples, the deformable flange 220 may extend at least partially around a portion of the dust cap body 46. The deformable flange 220 may have a first end 222 connected to the dust cap body 46 and a second free end 224. In some examples, the second free end extends radially outward in a distal direction. In some examples, the second end 224 of the deformable flange 220 may be configured to be received within and engage with the first and second flanges 38, 40 of the first and second legs 20, 22 of the connector element 12 (shown in FIG. 5) when the dust cap 14 is engaged with the connector element 12. When the dust cap 14 is connected to the connector element 12, the arrangement of the second end 224 of the deformable flange 220 within the space of the first and second flanges 39, 40 holds the dust cap 14 within to the connector element 12. By pulling the dust cap 14 in a distal direction, such as by pulling the grasping tab 58, the deformable flange 220 is configured to deform by the second end 224 folding over in a proximal direction (shown in FIG. 32B), for example when the second end 224 of deformable flange 220 pushes against the first and second flanges 38, 40 of the first and second legs 20, 22 of the connector element 12 as the dust cap 14 is moved in the distal direction and removed from engagement with the first and second legs 20, 22 during removal of the dust cap 14 from the connector element 12. Tampering with, potential contamination of, or previous use of the syringe 16 may be indicated by a deformed state of the deformable flange 220 on the dust cap 14 shown in FIG. 32B.

While various examples of the present disclosure were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A fluid path connector assembly for a medical fluid delivery system, the fluid path connector assembly comprising:
a connector element comprising:
a body comprising a perimeter skirt surrounding the connector element;
a lumen extending through the body;
a first flexible leg connected to the body, the first flexible leg having a first flange; and
a second flexible leg connected to the body, the second flexible leg having a second flange; and
a dust cap removably connected to the connector element, the dust cap comprising:
a dust cap body positioned between the first flexible leg and the second flexible leg and surrounding the body of the connector element to enclose the lumen, the dust cap body having a proximal end and distal end; and
an engagement element at the distal end of the dust cap body, the engagement element comprising a sloping member defined by a distal edge of the dust cap body,
wherein the engagement element is in contact with the first flange of the first flexible leg and the second flange of the second flexible leg of the connector element to prevent disengagement of the dust cap from the connector element, and
wherein the first flange of the first flexible leg and the second flange of the second flexible leg of the connector element are configured to contact the sloping member when the dust cap is moved distally relative to the connector element such that the first flexible leg and the second flexible leg expand radially outward to permit removal of the dust cap from the connector element.

2. The fluid path connector assembly according to claim 1, wherein the sloping member extends around at least a portion of an outer perimeter of the distal edge of the dust cap body.

3. The fluid path connector assembly according to claim 1, wherein the sloping member is a rounded circumferential edge or a chamfered circumferential edge.

4. (canceled)

5. The fluid path connector assembly according to claim 1, wherein the engagement element comprises a ledge configured to contact the first flange of the first flexible leg and the second flange of the second flexible leg of the connector element.

6. The fluid path connector assembly according to claim 1, further comprising a grasping tab connected to the distal end of the dust cap body.

7. (canceled)

8. (canceled)

9. The fluid path connector assembly according to claim 1, wherein, when the dust cap is connected to the connector element, air pressure within the lumen of the connector element may equalize with the outside atmospheric pressure while maintaining a sterility within the lumen of the connector element.

10. The fluid path connector assembly according to claim 1, wherein a tortuous fluid path is defined between the dust cap body and the circumferential skirt of the body of the connector element to permit equalization of pressure within the lumen of the connector element while preventing a passage of contaminants into the lumen of the connector element.

11. (canceled)

12. The fluid path connector assembly according to claim 1, further comprising a removeable/disconnectable tamper resistant or tamper evident feature connecting the dust cap to the connector element.

13. (canceled)

14. A fluid path connector assembly for a medical fluid delivery system, the fluid path connector assembly comprising:
a connector element comprising a body comprising a circumferential skirt surrounding the connector element, a lumen extending through the body, a first flexible leg connected to the body, and a second flexible leg connected to the body;
a dust cap removably connected to the connector element, the dust cap comprising a dust cap body positioned between the first flexible leg and the second flexible leg and surrounding the body of the connector element to enclose the lumen; and
a removeable/disconnectable tamper resistant or tamper evident feature connecting the dust cap to the connector element,
wherein, when present, the tamper resistant or tamper evident feature is configured to at least one of prevent removal of the dust cap from the connector element, indicate prior tampering with the connection between the connector element and the dust cap, and prevent or indicate reinstallation of the dust cap onto the connector element after the dust cap is removed from the connector element.

15. The fluid path connector assembly according to claim 14, wherein the tamper resistant or tamper evident feature comprising a polymeric shrink wrap or a paper wrap at least partially enclosing the connector element and the dust cap, the polymeric shrink wrap or the paper wrap having at least one removal feature for removing the shrink wrap from the fluid path connector assembly.

16. The fluid path connector assembly of claim 15, wherein the removal feature comprises a tearable pre-cut, a gripable tab, or perforation feature for removing the tamper resistant or tamper evident feature.

17. The fluid path connector assembly according to claim 14, wherein the tamper resistant or tamper evident feature comprises a cap at least partially enclosing the connector element and the dust cap, and wherein the cap is secured to a syringe via a tearable strip.

18. (canceled)

19. The fluid path connector assembly according to claim 14, wherein the tamper resistant or tamper evident feature comprises a first portion connected to the connector element and a second portion connected to the first portion and the dust cap,
wherein the first portion comprises a pull tab configured for removing the first portion from the connector element or wherein the second portion is configured to break from the first portion or the dust cap with rotation of the dust cap about a longitudinal axis.

20. (canceled)

21. (canceled)

22. The fluid path connector assembly according to claim 14, wherein the tamper resistant or tamper evident feature is a removable band surrounding the first flexible leg and the second flexible leg, and wherein the band restricts movement of the first flexible leg and the second flexible leg to prevent removal of the dust cap when the removable band is attached to the fluid path connector assembly.

23. The fluid path connector assembly according to claim 14, wherein the tamper resistant or tamper evident feature has a first end connected to the connector element and a second end connected to the dust cap, and wherein the first end is configured to break or separate from the connector element with rotation of the dust cap about a longitudinal axis.

24. The fluid path connector assembly according to claim 14, wherein the tamper resistant or tamper evident feature comprises a tearable pull tab monolithically formed with the dust cap.

25. (canceled)

26. (canceled)

27. The fluid path connector assembly according to claim 14, wherein the dust cap body comprises a first portion and a second portion connected together and separable from each other along a separation line substantially parallel with a longitudinal axis of the dust cap and wherein the first portion and the second portion have at least one reinsertion prevention element to prevent reinstallation of the dust cap on the connector element after the dust cap is removed from the connector element.

28. (canceled)

29. (canceled)

30. The fluid path connector assembly according to claim 14, wherein, when the dust cap and the tamper resistant or tamper evident feature are connected to the connector element, air pressure within the lumen of the connector element may equalize with the outside atmospheric pressure while maintaining a sterility within the lumen of the connector element.

31. (canceled)

32. (canceled)

33. The fluid path connector assembly according to claim 14, further comprising an engagement element at the distal end of the dust cap body, the engagement element comprising a sloping member defined by a distal edge of the dust cap body.

34. The fluid path connector assembly according to claim 33, wherein the engagement element is in contact with the first flange of the first flexible leg and the second flange of the second flexible leg of the connector element to prevent disengagement of the dust cap from the connector element, and
wherein the first flange of the first flexible leg and the second flange of the second flexible leg of the connector element are configured to contact the sloping member when the dust cap is moved distally relative to the connector element such that the first flexible leg and the second flexible leg expand radially outward to permit removal of the dust cap from the connector element.

35. The fluid path connector assembly according to claim 33, wherein the sloping member extends around at least a portion of an outer perimeter of the distal edge of the dust cap body.

36. The fluid path connector assembly according to claim 33, wherein the sloping member is a rounded circumferential edge or a chamfered circumferential edge.

37. (canceled)

38. (canceled)
